(19) United States
(12) Patent Application Publication
(10) Pub. No.: US 2013/0244909 A1
(43) Pub. Date: Sep. 19, 2013

(54) METHODS AND APPARATUS FOR CLASSIFICATION AND QUANTIFICATION OF MULTIFUNCTIONAL OBJECTS

(71) Applicant: FIREFLY BIOWORKS, INC., Cambridge, MA (US)

(72) Inventors: Andreas Windemuth, South Glastonbury, CT (US); Daniel Pregibon, Cambridge, MA (US); Davide Marini, Cambridge, MA (US)

(21) Appl. No.: 13/790,662

(22) Filed: Mar. 8, 2013

Related U.S. Application Data
(60) Provisional application No. 61/609,244, filed on Mar. 9, 2012.

(51) Int. Cl. G01N 33/50; G06M 3/08

(52) U.S. Cl. CPC G01N 33/50; G06M 3/08
USPC 506/39; 435/288.7; 422/68.1; 422/82.05; 422/82.08; 702/21

(57) ABSTRACT

A method may include accessing data regarding a number of events, where the events were detected by a particle detection apparatus, and identifying a number of groups in the events. Each of the groups includes two or more events, and each event includes one or more of a time stamp, a time duration, a fluorescence intensity, a scatter measurement, a radio frequency signal, a magnetic signal, a neutron scattering measurement, a light scattering measurement, an electron scattering measurement, an audio signal, an acoustic signal, a mechanical signal, an electrical resistance, a thermal property, and a width of fluorescence signal. The method may include identifying a subset of the groups as a number of objects detected by the particle detection apparatus, where each object is identified based at least in part upon one or more quantities, where each quantity is identified by or derived from the respective two or more events.

### RELATED APPLICATIONS

The present application claims priority to U.S. Provisional Application 61/609,244 entitled “Classification and Fluorescence Quantification of Multi-Region Particles” and filed Mar. 9, 2012, the contents of which are hereby incorporated by reference in its entirety.

### BACKGROUND

In biological, clinical and diagnostic research the need exists to identify a set of biomolecules that might be present in the same sample, at a specific point in time. A typical solution to multiplexed detection is to mix a sample with encoded particles, each of which is functionalized with a probe that will recognize a specific target, and then analyze results from each of those particles. For this method to work reliably, it is important to (1) encode each particle so it can be reliably identified and to (2) quantify the amount of target bound to each particle. An example of such solution relies on color-coded particles that are interrogated by scanning them in a flow cytometer. In this approach, encoding is achieved by using precisely-controlled levels of various fluorochromes embedded in the particles, while target quantification is achieved by measuring the fluorescence intensity of a fluorophore bound to the target, which is in turn bound to the particle's surface. In this method, code identification and target quantification are performed contemporaneously. In other words, both the encoding and the target-bound dyes are located in the same place, so their signals need to be de-convoluted by sophisticated optical or electronic methods. For this reason, higher multiplexing requires more expensive equipment.

### SUMMARY

The present disclosure describes embodiments of an apparatus and method for interpreting the data (e.g., raw signal data) provided by a particle detection apparatus, such as a flow cytometer. In further examples, particle detection apparatus may include particle counters, Coulter counters, microarray scanners, or plate imagers. In some implementations, the data may be acquired in the process of reading a universally coded particle (UCP) assay. In a broader sense, these techniques apply to any multifunctional particle or other object, such as a biological object (e.g., DNA strand, RNA strand, cell, worm, etc.), photolithographically shaped hydrogel particles, as well as any other sort of shaped particles, particularly also nanostructures and protein, RNA, or DNA aggregates (e.g., DNA origami, various other means of creating nanostructures). UCP assays are advantageous because they can be read in standard cytometers with no need for dedicated instrumentation. In some embodiments, multifunctional objects include one or more active (e.g., encoded, signal-generating, etc.) regions. For example, universally coded particles (UCP) may be made as linear rod-like structures with one or more active (e.g., encoded, signal-generating, etc.) regions. Active regions, for example, may contain controlled amounts of fluorophores or other elements that can be detected in a cytometer or other flow device. In a particular embodiment, a UCP may include three active regions, for example as shown in FIG. 1. The number of active regions, as used herein, is denoted in the following as Nr.

In some embodiments, data output obtained by scanning or otherwise measuring multifunctional objects can be interpreted to identify one or more codes (e.g., discrete levels, patterns, or other signals identifiable via measurement data obtained by the particle detection apparatus). The interpreted data may be transformed into a useful format, for example for presentation to a user of a computing device including a display or as a printable report.

A standard cytometer is equipped to create a single-file stream of particles in a fluid, detect a triggering event to record data, and measure a series of signals, such as fluorescence, associated with the particles. The signal from each particle may be detected in one or multiple detection channels. Many different wavelength bands can be configured, for example to match the many different fluorophores that can be used. Typical laser excitation wavelengths include blue (e.g., approximately 488 nm), green (e.g., approximately 532 nm) and red (e.g., approximately 633 nm) with detection channels of red (R, approximately 660 to 690 nm), yellow (Y, approximately 580 nm), and green (G, approximately 530 nm). In addition to fluorescence, in some implementations, scattered light may also measured, normally in two channels: forward scatter (FSC) and side scatter (SSC). In some embodiments, one of the detection channels may be selected as the trigger channel (TRG), where all active regions of the particle are configured to emit a signal above a designated threshold level in the trigger channel. As particles pass by the scanning region of the cytometer, for example, a signal in the trigger channel may cause the cytometer to record one event. In some implementations, an event includes a time value (TIME) and one or more signal measurements, such as detected light intensities for all the configured detection channels. In some implementations, other information, such as the duration of the signal (WIDTH), may be recorded.

Using a set of algorithms, in some implementations, data included in the standard output of a flow cytometer (e.g., obtained when scanning multifunctional objects) may be interpreted for use in multiplexed detection of biomolecules or other entities. Using these algorithms, for example, the multiple events recorded for each multifunctional object may be appropriately grouped together and recognized as pertaining to the same object. The algorithms may be used to reconstruct each multifunctional object, in some implementations, revealing a code associated with the particular object and measuring a probe signal of the object. The interpreted data output by the algorithms may then be provided to a user. For example, the interpreted data may be represented in a user interface, where the interpreted data can be visualized, manipulated, and saved.

This application is related to International Patent Application number PCT/US11/39529 entitled “Scanning Multifunctional Particles” and filed Jun. 7, 2011; International Patent Application number PCT/US11/39531 entitled “Nucleic Acid Detection and Quantification by Post-Hybridization Labeling and Universal Encoding” and filed Jun. 7, 2011; U.S. provisional patent application Ser. No. 61/352,018, filed Jun. 7, 2010, Ser. No. 61/365,738, filed Jul. 19, 2010, and Ser. No. 61/387,958, filed Sep. 29, 2010, the entire contents of which are herein incorporated by reference in their entireties.

In one aspect, the present disclosure relates to a method including accessing data regarding a number of events, where the number of events were detected by a particle detection apparatus, and identifying, by a processor of a computing device, a number of groups in the number of events. Each of the number of groups includes two or more events, and each event of the number of events includes one or more of a time stamp, a time duration, a fluorescence intensity, a scatter measurement, a radio frequency signal, a magnetic signal, a neutron scattering measurement, a light scattering measurement, an electron scattering measurement, an audio signal, an acoustic signal, a mechanical signal, an electrical resistance, a thermal property, and a width of fluorescence signal. The method may include identifying, by the processor, a subset of the number of groups as a number of objects detected by the particle detection apparatus, where each object of the number of objects is identified based at least in part upon one or more quantities, where each quantity of the one or more quantities is identified by or derived from the respective two or more events.

In some embodiments, the number of objects include at least one of a number of cells, a number of DNA fragments, a number of RNA fragments, a number of protein aggregates, a number of nanostructures, and a number of living organisms. Each object of the number of objects may be composed at least in part of one or more of a) hydrogel, b) metal, c) glass, and d) plastic.

In some embodiments, the number of objects include a number of encoded objects. Each event of the number of events may include one or more measurements, where the one or more measurements were obtained by the particle detection apparatus, and a first measurement of the one or more measurements includes a measurement of a signal encoded to emanate from each object of at least a portion of the number of encoded objects. Identifying a first group of the number of groups may include identifying at least a first region of a particular object and a second region of the particular object, where a number of signals emanate from two or more spatially separated regions of the particular object.

In some embodiments, the number of encoded objects includes a first object type and a second object type. Identifying the subset of the number of groups may include identifying a second subset of the number of groups, where at least one region of the encoded objects of the first subset of the number of groups varies in one or more physical characteristics from a corresponding region of the encoded objects of the second subset of the number of groups, where the at least one region varies at a discrete level, allowing the first object type to be reliably distinguished from the second object type based upon the data. The signal may include a light signal. The method may further include quantifying a signal associated with a third region of the particular object, where the third region is a probe region of the particular object. The third region may be the first region.

In some embodiments, identifying the first group includes one or more of: (a) comparing a time interval between a pair of events of the number of events with an expected interval, where the expected interval is based at least in part on a combination of (i) a flow velocity setting of the particle detection apparatus at time of detection, and (ii) a physical distance between a pair of event sources on the particular particle; (b) comparing the duration of a first event of the number of events with an expected duration, where the expected duration is based at least in part on a combination of i) the velocity setting of the particle detection apparatus at time of detection, and (ii) a physical dimension of a first event source on the particular particle; (c) comparing fluorescence intensities of a sequence of two events of the number of events with an expected sequence of fluorescence intensities, where the expected sequence of fluorescence intensities is based at least in part on optical characteristics of the particular particle; and (d) comparing scattering intensities of a sequence of two events of the number of events with an expected sequence of scattering intensities, where the expected sequence of scattering intensities is based at least in part on optical characteristics of the particular particle.

In some embodiments, identifying a first object of the number of objects includes: (a) defining a fit-function F of a number of measurements, where the number of measurements are obtained from the number of events, and the fit-function F is configured to evaluate the correspondence of each event of the number of events with known physical characteristics of the particular particle; (b) selecting, from the number of events, a subset of the number of events which optimizes the fit-function F, where the subset of the number of events is selected as a particular group of events most likely to originate from a same physical object of the number of objects; and (c) assigning a score to a fit identified by the fit-function F, where the score is configured to assess a probability of error in selecting the correct subset of the number of events.

In some embodiments, the fit-function F is the root-mean-square difference between observed and expected measurements:

$$F = {\sum }_{i = 1}^{{N}_{r}} {( {M}_{i} - {E}_{i} )}^{2} ,$$

where Nr is a number of events per group, values Mi include the measurements of a single quantity for the candidate combination of events, and values Ei include the expected measurements of this quantity based upon a model assuming that all events belong to a same object. The number of events may include measurements of two or more quantities, and a combined fit function

$$F = {\sum }_{j = 1}^{N} {F}_{j}$$

may be used to evaluate the subset of the number of groups. Each of the Mᵢ and Eᵢ may be calculated as a mathematical function of two or more measured quantities for each event. The mathematical function may be a linear combination. Coefficients of the linear combination may be selected to account for an amount of bleed-through between different fluorescent dyes or optical channels. The method may include inferring, from the data, the amount of bleed-through, where the amount of bleed-through is inferred from the data by comparing measured quantities with physical characteristics of the objects.

In some embodiments, the mathematical function is a ratio. The method may further include selecting the two or more measured quantities as being equally affected by variation in measurement during detection by the particle detection apparatus, such that said variation is reduced in the ratio. The particle detection apparatus may include one or more light sources, and the two or more measured quantities may include fluorescence signals, where the fluorescence signals emanate from fluorophores excited by a same light source of the one or more light sources.

In some embodiments, identifying the number of groups includes: (a) out of a first Nᵣ+g consecutive unassigned events of the number of events, starting with a consecutive event following a last unassigned event of the number of events, selecting all combinations of Nᵣ events, where a gap count g indicates a number of allowed gaps, where the gap count g is configured to range from zero to any positive integer; (b) calculating, for each selected combination of Nᵣ events, the fit function F with respective candidate regions of respective candidate combinations of events assigned to events in order of increasing time to identify respective forward direction fits; (c) calculating, for each selected combination of Nᵣ events, the fit function F with respective candidate regions of respective candidate combinations of events assigned to events in order of decreasing time to identify respective reverse direction fits; (d) identifying, from the forward direction fits and the reverse direction fits, i) a lowest fit combination of the selected combination of Nᵣ events and ii) a respective direction of the lowest fit combination, where the events of the lowest fit combination are assigned to respective regions of the object according to the direction of the lowest fit combination; and (e) repeating steps (a) through (d) until a remaining number of events after the last assigned event is less than Nᵣ.

In some embodiments, the method includes identifying an orientation of each object of the number of objects. The particle detection apparatus may include standard flow cytometry instrumentation. The data may include a file in the standard flow cytometry format (FCS).

In one aspect, the present disclosure relates to a system including a particle detection apparatus, a processor, and a memory storing instructions. The instructions, when executed, may cause the processor to access data regarding a number of events, where the number of events were detected by the particle detection apparatus, and identify a number of groups in the number of events, where each of the number of groups includes one or more events, and a first event of the one or more events includes at least one measurement selected from the group consisting of: a time duration, a fluorescence intensity, a scatter measurement, a radio frequency signal, a magnetic signal, a neutron scattering measurement, a light scattering measurement, an electron scattering measurement, an audio signal, an acoustic signal, a mechanical signal, an electrical resistance, a thermal property, and a width of fluorescence signal, where the particular detection apparatus collected the measurement. The instructions may cause the processor to identify, based at least in part on the at least one measurement associated with each group of the number of groups, a subset of the number of groups as a number of objects.

In some embodiments, a first measurement of the at least one measurement may include a measurement of a signal encoded to emanate from each object of at least a portion of the number of encoded objects. Identifying a first group of the number of groups may include identifying at least a first region of a particular object and a second region of the particular object, where a number of signals emanate from two or more spatially separated regions of the particular object.

In some embodiments, the number of encoded objects include a first object type and a second object type. Identifying the subset of the number of groups may include identifying a second subset of the number of groups, where at least one region of the objects of the first subset of the number of groups varies in one or more physical characteristics from a corresponding region of the objects of the second subset of the number of groups, where the at least one region varies at a discrete level, allowing the first object type to be reliably distinguished from the second object type based upon the data. Two or more predetermined sets of levels may be combined into codes for encoding the number of objects, thereby allowing a number of different sets of objects to be identified.

In some embodiments, the number of objects include a number of carriers brought in contact with a sample including an analyte prior to detection by the particle detection apparatus. The analyte may include a protein or a nucleic acid. One or more of the measurements associated with each object of the number of objects may be indicative of a concentration of the analyte within the sample. Identifying the subset of the number of groups as the number of objects may include identifying the number of objects as being a type of object sensitive to the analyte. The instructions, when executed, may further cause the processor to determine the concentration of the analyte, where the concentration of the analyte is determined by statistical analysis of the one or more measurements associated with each object of the number of objects. Two or more different carriers may be brought in contact with the sample simultaneously, and determining the concentration of the analyte may include determining respective concentrations of two or more analytes.

In some embodiments, the statistical analysis includes the calculation of one or more of the following: mean, median, standard deviation and confidence intervals. The instructions may further cause the processor to, prior to determining the concentration of the analyte: identify one or more outlier measurements of the number of measurements associated with the number of objects, and remove the one or more outlier measurements from a set of measurements provided for statistical analysis. Identifying the one or more outlier measurements may include ordering all measurements, and selecting a lower percentile and upper percentile.

The instructions, when executed, may cause the processor to determine, for each object of the number of objects, based in part upon respective one or more quantities associated with the respective object, information regarding a history of the respective object. The history of the respective object may be determined at least in part by a physical, chemical or biological assay.

In one aspect, the disclosure relates to a non-transitory computer-readable medium having instructions stored thereon, where the instructions, when executed by a processor, cause the processor to access data regarding a number of events, where the number of events were detected by a particle detection apparatus, and identify a number of groups in the number of events, where each of the number of groups includes one or more events, and a first event of the one or more events includes at least one measurement selected from the group consisting of: a time duration, a fluorescence intensity, a scatter measurement, a radio frequency signal, a magnetic signal, a neutron scattering measurement, a light scattering measurement, an electron scattering measurement, an audio signal, an acoustic signal, a mechanical signal, an electrical resistance, a thermal property, and a width of fluorescence signal, where the particular detection apparatus collected the measurement. The instructions, when executed, may cause the processor to identify, based at least in part on the at least one measurement associated with each group of the number of groups, a subset of the number of groups as a number of objects.

### BRIEF DESCRIPTION OF THE FIGURES

The drawings are for illustration purposes only, not for limitation.

FIG. 1 illustrates an example system for classification and quantification of multifunctional objects;

FIGS. 2A and 2B illustrate a conceptual example of how scanning of multifunctional particles could be implemented in some embodiments;

FIGS. 3 and 4 illustrate an example graphical user interface screens for presenting interpreted data;

FIG. 5A illustrates representative raw data obtained when scanning universally coded particles on a flow cytometer;

FIG. 5B illustrates an example graphical user interface screen presenting an interpretation of the raw data of FIG. 5A;

FIG. 6 illustrates an example flow chart of a data analysis process;

FIG. 7 is a block diagram of an example network environment; and

FIG. 8 is a block diagram of a computing device and a mobile computing device.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DEFINITIONS

In order for the present disclosure to be more readily understood, certain terms are first defined below. Additional definitions for the following terms and other terms are set forth throughout the specification.

“Analyte”: As used herein, the term “analyte” broadly refers to any substance to be analyzed, detected, measured, or quantified. Examples of analytes include, but are not limited to, proteins, peptides, hormones, haptens, antigens, antibodies, receptors, enzymes, nucleic acids, polysaccharides, chemicals, polymers, pathogens, toxins, organic drugs, inorganic drugs, cells, tissues, microorganisms, viruses, bacteria, fungi, algae, parasites, allergens, pollutants, and combinations thereof.

“Biomolecules”: The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

“Encoding region,” “coding region,” or “barcoded region”: As used herein, the terms “encoding region,” “coding region,” “barcoded region”, or grammatical equivalents, refer to a region on an object or substrate (e.g., particle) that can be used to identify the object or substrate (e.g., particle). These terms may be used inter-changeably. Typically, an encoding region of an object bears graphical and/or optical features associated with the identity of the object. Such graphical and/or optical features are also referred to as signature features of the object. In some embodiments, an encoding region of an object bears spatially patterned features (e.g., stripes with various shapes and/or dimensions, or a series of holes with various sizes) that give rise to variable fluorescent intensities (of one or multiple wavelengths). In some embodiments, an encoding region of an object bears various type and/or amount of fluorophores or other detectable moieties, in various spatial patterns, that give rise to variable fluorescent signals (e.g., different colors and/or intensities) in various patterns.

“Labeled”: The terms “labeled” and “labeled with a detectable agent or moiety” are used herein interchangeably to specify that an entity (e.g., a nucleic acid probe, antibody, etc.) can be visualized, for example following binding to another entity (e.g., a nucleic acid, polypeptide, etc.). The detectable agent or moiety may be selected such that it generates a signal which can be measured and whose intensity is related to (e.g., proportional to) the amount of bound entity. A wide variety of systems for labeling and/or detecting proteins and peptides are known in the art. Labeled proteins and peptides can be prepared by incorporation of, or conjugation to, a label that is detectable by spectroscopic, photochemical, biochemical, immunochemical, electrical, optical, chemical or other means. A label or labeling moiety may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody including a reporter such as a fluorophore). Suitable detectable agents include, but are not limited to, radionucleotides, fluorophores, chemiluminescent agents, microparticles, nanoparticles, enzymes, colorimetric labels, magnetic labels, haptens, molecular beacons, aptamer beacons, and the like.

“Particle”: The term “particle,” as used herein, refers to a discrete object. Such object can be of any shape or size. Composition of particles may vary, depending on applications and methods of synthesis. Suitable materials include, but are not limited to, plastics, ceramics, glass, polystyrene, methylstyrene, acrylic polymers, metal, paramagnetic materials, thoria sol, carbon graphited, titanium dioxide, latex or cross-linked dextrans such as Sepharose, cellulose, nylon, cross-linked micelles and teflon. In some embodiments, particles can be optically or magnetically detectable. In some embodiments, particles contain fluorescent or luminescent moieties, or other detectable moieties. In some embodiments, particles having a diameter of less than 1000 nanometers (nm) are also referred to as nanoparticles. Particles, in some implementations, are self-assembling aggregates of biological or nonbiological polymers, such as DNA origami.

“Probe”: As used herein, the term “probe” refers to a fragment of DNA or RNA of variable length (e.g., 3-1000 bases long), which is used to detect the presence of target nucleotide sequences that are complementary to the sequence in the probe. Typically, the probe hybridizes to single-stranded nucleic acid (DNA or RNA) whose base sequence allows probe-target base pairing due to complementarity between the probe and target. More generally, a probe can be any entity that is suitable to respond to a specific property of a sample (such as presence of other entities) by the emission or transmission of some sort of signal. In addition to DNA or RNA, antibodies are often used as probes. The probe is included in a probe region of the particle or object. Each particle or object includes one or more probe regions for sample quantification.

“Signal”: As used herein, the term “signal” refers to a detectable and/or measurable entity. In certain embodiments, the signal is detectable by the human eye, e.g., visible. For example, the signal could be or could relate to intensity and/or wavelength of color in the visible spectrum. Non-limiting examples of such signals include colored precipitates and colored soluble products resulting from a chemical reaction such as an enzymatic reaction. In certain embodiments, the signal is detectable using an apparatus. In some embodiments, the signal is generated from a fluorophore that emits fluorescent light when excited, where the light is detectable with a fluorescence detector. In some embodiments, the signal is or relates to light (e.g., visible light and/or ultraviolet light) that is detectable by a spectrophotometer. For example, light generated by a chemiluminescent reaction could be used as a signal. In some embodiments, the signal is or relates to radiation, e.g., radiation emitted by radioisotopes, infrared radiation, etc. In certain embodiments, the signal is a direct or indirect indicator of a property of a physical entity. For example, a signal could be used as an indicator of amount and/or concentration of a nucleic acid in a biological sample and/or in a reaction vessel.

### DETAILED DESCRIPTION OF CERTAIN EMBODIMENTS

Disclosed herein, among other things, are methods and systems for characterizing multifunctional objects using a flow-through device, such as, a flow cytometer. An inventive method according to an illustrative embodiment includes one or more steps of (a) interrogating a plurality of objects (e.g., particles), wherein each individual object (e.g., particle) includes one or more interrogation regions detectable as a sequence of events; (b) recording multiple events, wherein each individual event corresponds to each individual interrogation region detectable above a pre-determined triggering threshold; (c) grouping the recorded multiple events, and (d) characterizing the plurality of objects based on the grouped events. In some embodiments, the multiple events are recorded non-contemporaneously. In some embodiments, each interrogation region is characterized by a detectable signal pattern once interrogated. In some embodiments, the recorded events or signal patterns may be grouped based on spatial and/or temporal-proximity. In some embodiments, the recorded events or signal patterns may be grouped based on patterns of measured properties.

Certain embodiments of the present disclosure are particularly useful for multiplexed analyte detection and/or quantification. According some embodiments, the binding between one or more target analytes and one or more objects (e.g., particles) typically alters events or signal patterns detected by inventive methods described herein. Therefore, the presence of the one or more target analytes may be detected based on the altered patterns. In some embodiments, the amount of analytes bound to objects (e.g., particles) may be further quantified based on the level of alteration.

Thus, the present disclosure provides compositions, methods and systems that permit multiplexed, robust, and efficient detection and/or quantification of target analytes based on rapid flow-through particle scanning using simple, inexpensive, or portable devices.

Various aspects of methods and apparatus for classification and quantification of multifunctional objects are described in further detail in the following subsections. The use of subsections is not meant to limit the scope of embodiments. Each subsection may apply to any aspect of the methods and apparatus described herein. In this application, the use of “or” means “and/or” unless stated otherwise.

The teachings herein may be used to characterize any objects. Suitable objects include, but are not limited to, particles, beads, phages (e.g., phages suitable for phage display), macromolecules (e.g., proteins including peptides or aggregated peptides, DNAs including DNA origami, and/or RNAs), cells including any genetically engineered cells (e.g., cells carrying green fluorescent protein (GFP) derivatives thereof and the like), micro-organisms (e.g., C. elegans (e.g., engineered nematodes for drug testing), bacteria, yeast, and/or fungi) including any genetically engineered micro-organisms (e.g., micro-organisms carrying GFP derivatives thereof and the like).

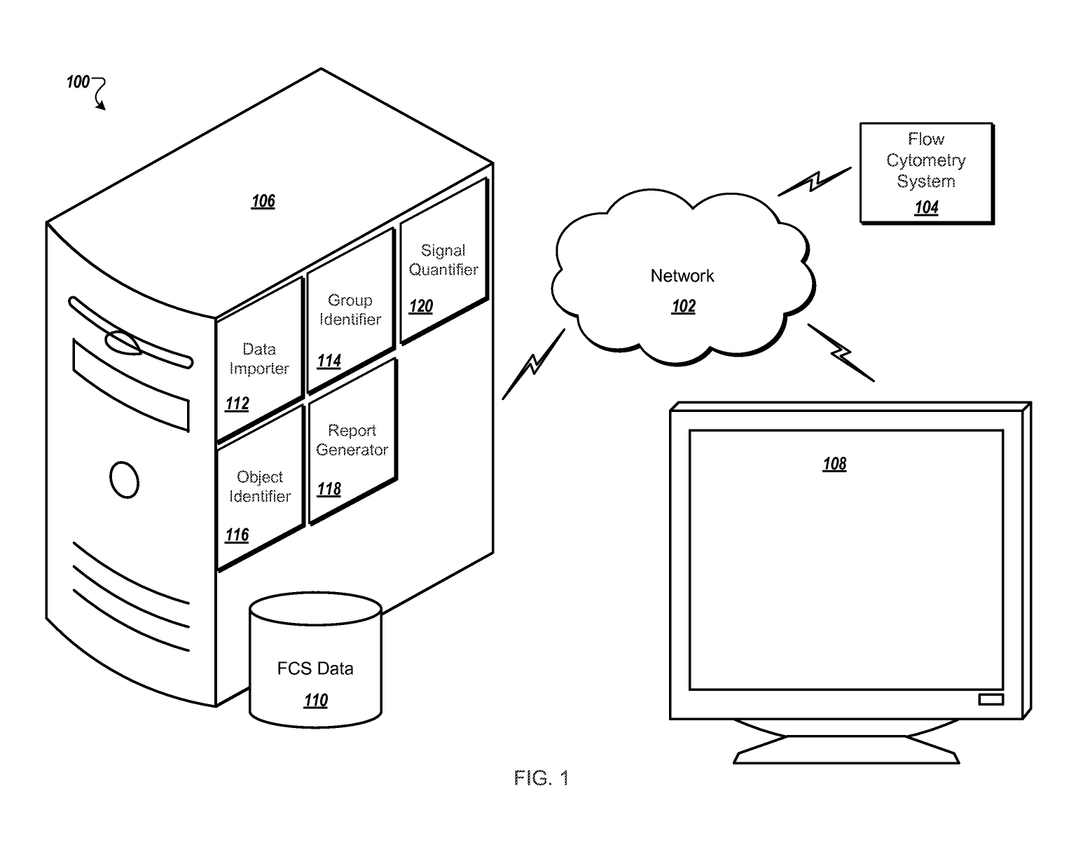

Turning to FIG. 1, in an example system 100 for characterizing multifunctional objects, flow cytometry system (FCS) data 110, in some implementations, is collected by a flow cytometry system 104 and provided to an analysis system 106 (e.g., via a network 102 or a direct connection between computing systems). The analysis system 106, in some implementations, includes a data importer 112 for importing the FCS data 110. The data importer 112, for example, may reformat the data into a standardized format used by the analysis system 106. If, for example, data is imported from a variety of particle detection apparatus, the analysis system 106 may accept raw data in a variety of formats and convert the data for further analysis.

In some implementations, the analysis system 106 includes a group identifier 114 for identifying groups of events within the FCS data 110. The groups of events, for example, may be based in part upon one or more measurements such as, in some examples, a time stamp, a time duration, a fluorescence intensity, a scatter measurement, a radio frequency signal, a magnetic signal, a neutron scattering measurement, a light scattering measurement, an electron scattering measurement, an audio signal, and/or a width of fluorescence signal. In some implementations, a group may include two or more events. The group, in some implementations, may be identified in either orientation (e.g., forwards or backwards). In some implementations, the groups of events are identified based upon one or more known object fingerprints (e.g., types or patterns of measurements associated with an expected object). The group identifier 114 may identify two or more types of groupings within the FCS data 110.

In some implementations, the analysis system 106 includes an object identifier 116 for identifying objects within the identified groups of events. For example, the object identifier 116 may apply one or more of a threshold value analysis, a scoring function, a fit function, and a probability analysis to identify objects from the groups of events. For example, the groups of events may be considered to be prospective objects (e.g., measurements that may be indicative of an object). The object identifier 116 analyzes the groups of events to identify measurement patterns indicative of a type of object. In some implementations, the object identifier 116 analyzes the groups to identify two or more types of objects. For example, the sample provided to the flow cytometry system 104 may have included two or more objects, each with a different fingerprint (e.g., type and/or pattern of event grouping). The object identifier 116 may analyze groups of events to identify both likely objects and the most likely type of object attributed to groups of events.

In some implementations, the analysis system 106 includes a signal quantifier 120 for quantifying signals measured by the flow cytometry system 104 in relation to discrete objects. The signal quantifier 120 combines the signals from multiple objects of the same code (e.g., objects including a same type of code region) into an integrated single best estimate of the property. For example, the signal may be a signal that a particular assay is designed to measure. Any method of statistical inference, for example, may be used to quantify an identified signal. The quantification of signals related to the identified objects is described in greater detail below in the section entitled “Signal Quantification.”

In some implementations, the analysis system 106 includes a report generator 118 for generating report data for presentation on a display device 108. For example, the report generator 118 may prepare graphical user interface data including one or more of particle clusters, signal ranges, a number of objects identified, types of objects identified, confidence factors associated with the identification of objects, event collection information associated with the flow cytometry system 104 (e.g., equipment settings, equipment sensitivity, equipment type, etc.) and sample information associated with the FCS data 110. The graphical user interface may include a variety of graph analysis for presenting the analyzed FCS data 110.

Although illustrated as separate systems, in other implementations, two or more of the flow cytometry system 104, the analysis system 106, and the display 108 may be combined within a single system. In some implementations, at least a portion of the analysis system 106 may be included within the flow cytometry system 104. For example, the group identifier 114 may be included within the flow cytometry system 104, while the report generator 118 may be a separate software system. Other variations are possible.

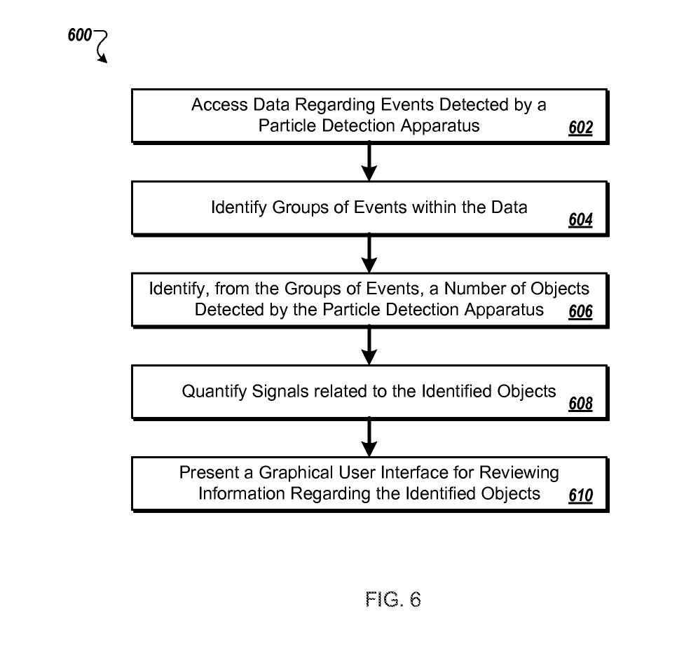

Turning to FIG. 6, in some implementations, a process 600 for classification and quantification of multifunctional objects begins with accessing data regarding events detected by a particle detection apparatus (602). The FCS data 110, for example, obtained by the flow cytometry system 104, may be accessed by the analysis system 106, as described in relation to FIG. 1. The multifunctional particles may include one or more types of multifunctional particles or other object, such as a biological object (e.g., DNA strand, RNA strand, cell, worm, etc.). Examples of types of objects and/or particles are listed in greater detail below in the section entitled “Objects and/or Particles”. The events detailed within the data may be acquired, in some examples, by a flow cytometer, particle counter, Coulter counter, microarray scanner, or plate imager. In some implementations, the data may be acquired in the process of reading a universally coded particle (UCP) assay. The data, in a particular example, may be recorded in a standard flow cytometry data file, formatted according to the FCS standard. In some implementations, a raw data file may be parsed to extract an array of data. For example, the raw data file may be parsed to identify an array of data including a single numerical value for each event and channel combination.

In some implementations, groups of events are identified within the data (604). For example, as described in relation to FIG. 1, the group identifier 114 may be used to identify groups of events within the FCS data 110. In some implementations, the recorded events or signal patterns may be grouped based on spatial and/or temporal-proximity. In some implementations, the recorded events or signal patterns may be grouped based on patterns of measured properties. The grouping of events is described in greater detail below in the section entitled “Object Recognition”.

In some implementations, a number of objects detected by the particle detection apparatus are identified from the groups of events (606). The object identifier 116, for example, may be used to identify objects within the FCS data 110 from the groups identified by the group identifier 114, as described in relation to FIG. 1. Identification of objects is described in greater detail below in the section entitled “Object Identification.”

In some implementations, signals related to the identified objects are quantified (608). The signal quantifier 120, for example, may be used to quantify signals measured by the flow cytometry system 104, as described in relation to FIG. 1. Signal quantification is the step which combines the signals from multiple objects of the same code into an integrated single best estimate of the property. For example, the signal may be a signal that a particular assay is designed to measure. Any method of statistical inference, for example, may be used to quantify an identified signal. The quantification of signals related to the identified objects is described in greater detail below in the section entitled “Signal Quantification.”

In some implementations, a graphical user interface is presented for reviewing information regarding the identified objects (610). The report generator 118, for example, may be used to generate various graphical user interface views upon the display 108, as described in relation to FIG. 1. Example graphical user interfaces for reviewing, interpreting, and augmenting the object information are described in greater detail below in the section entitled “User Interface.”

Although described in a particular order, in some implementations, one or more of the steps of the process 600 may be performed in a different order. For example, in some implementations, a user may review and augment information via the graphical user interface (610) prior to quantification of the signals related to the identified objects (608). In some implementations, steps of the process 600 may be added or removed. For example, rather than presenting the graphical user interface (610), the data may be formatted for exportation (e.g., to a separate analysis system, to a spreadsheet tool, etc.). Other modifications to the process 600 are possible.

Objects and/or Particles

For illustration purposes, the terms particle and object may be interchangeable as described in connection with various embodiments below.

Particles suitable for use in accordance with various embodiments described herein can be made of any materials. Suitable particles can be biocompatible, non-biocompatible. Suitable particles can also be biodegradable or non-biodegradable.

In some embodiments, particles are made of polymers. Exemplary polymers include, but are not limited to, poly(arylates), poly(anhydrides), poly(hydroxy acids), polyesters, poly(ortho esters), poly(alkylene oxides), polycarbonates, polypropylene fumerates), poly(caprolactones), polyamides, polyamino acids, polyacetals, polylactides, polyglycolides, poly(dioxanones), polyhydroxybutyrate, polyhydroxyvalyrate, poly(vinyl pyrrolidone), polycyanoacrylates, polyurethanes and polysaccharides. In some embodiments, polymers of particles include polyethylene glycol (PEG). In some embodiments, polymers of particles may be formed by step or chain polymerization. The amount and kind of radical initiator, such as, in some examples, photo-active initiator (e.g., UV or infrared), thermally-active initiator, or chemical initiator, or the amount of heat or light employed, may be used to control the rate of reaction or modify the molecular weight. Where desired, a catalyst may be used to increase the rate of reaction or modify the molecular weight. For example, a strong acid may be used as a catalyst for step polymerization. Trifunctional and other multifunctional monomers or cross-linking agents may also be used to increase the cross-link density. For chain polymerizations, the concentration of a chemical initiator in a mixture of one or more monomers may be adjusted to manipulate final molecular weight.

Exemplary methods for making particles are described in U.S. Pat. No. 7,709,544 entitled “Microstructure Synthesis by Flow Lithography and Polymerization” and filed Oct. 25, 2006 and U.S. Pat. No. 7,947,487, entitled “Multifunctional Encoded Particles for High-Throughput Analysis” and filed Oct. 4, 2007, the entire contents of which are incorporated herein by reference. For example, processes as discussed can be conducted with any polymerizable liquid-phase monomer in which shapes of particles suitable for use in various embodiments described herein, can be defined and polymerized in a single lithography-polymerization step. Exemplary monomers include Allyl Methacrylate, Benzyl Methylacrylate, 1,3-Butanediol Dimethacrylate, 1,4-Butanediol Dimethacrylate, Butyl Acrylate, n-Butyl Methacrylate, Diethyleneglycol Diacrylate, Diethyleneglycol Dimethacrylate, Ethyl Acrylate, Ethyleneglycol Dimethacrylate, Ethyl Methacrylate, 2-Ethyl Hexyl Acrylate, 1,6-Hexanediol Dimethacrylate, 4-Hydroxybutyl Acrylate, Hydroxyethyl Acrylate, 2-Hydroxyethyl Methacrylate, 2-Hydroxypropyl Acrylate, Isobutyl Methacrylate, Lauryl Methacrylate, Methacrylic Acid, Methyl Acrylate, Methyl Methacrylate, Monoethylene Glycol, 2,2,3,3,4,4,5,5-Octafluoropentyl Acrylate, Pentaerythritol Triacrylate, Polyethylene Glycol (200) Diacrylate, Polyethylene Glycol (400) Diacrylate, Polyethylene Glycol (600) Diacrylate, Polyethylene Glycol (200) Dimethacrylate, Polyethylene Glycol (400) Dimethacrylate, Polyethylene Glycol (600) Dimethacrylate, Stearyl Methacrylate, Triethylene Glycol, Triethylene Glycol Dimethacrylate, 2,2,2-Trifluoroethyl 2-methylacrylate, Trimethylolpropane Triacrylate, Acrylamide, N,N,-methylene-bisacryl-amide, Phenyl acrylate, Divinyl benzene, etc. In certain embodiments, a monomer is characterized by a polymerization reaction that can be terminated with a termination species. The terminating species, lithographic illumination, and monomer constituents are therefore selected in cooperation to enable making particles suitable for use in classification and quantification of multifunctional objects.

In some embodiments, particles are hydrogels. In general, hydrogels include a substantially dilute crosslinked network. Water or other fluids can penetrate in the network forming such a hydrogel. In some embodiments, hydrogels suitable for use in various embodiments of the present disclosure are made of or include a hydrophilic polymer. For example, hydrophilic polymers may include anionic groups (e.g. phosphate group, sulphate group, carboxylate group); cationic groups (e.g. quaternary amine group); or polar groups (e.g. hydroxyl group, thiol group, amine group). In some embodiments, hydrogels are superabsorbent (e.g. they can contain over 99% water) and possess a degree of flexibility very similar to natural tissue, due to their significant water content. Both of weight and volume, hydrogels are fluid in composition and thus exhibit densities to those of their constituent liquids (e.g., water). The present disclosure encompasses the recognition that hydrogels are particularly useful in some embodiments of classification and quantification of multifunctional objects. Without wishing to be bound to any particular theory, it is contemplated that hydrogels enable 1) ease of implementation with detection instruments, in particular, commercially available instruments without substantial modifications (e.g., flow cytometers), and 2) ease of incorporation of functional moieties (e.g., in a single lithography-polymerization step) without requiring surface functionalization. Due to their bio-friendly nature, hydrogels have been used extensively in the fields of tissue engineering, drug delivery, and biomolecule separation.

Various additional materials and methods can be used to synthesize particles. In some embodiments, particles may be made of or include one or more polymers. Polymers used in particles may be natural polymers or unnatural (e.g. synthetic) polymers. In some embodiments, polymers can be linear or branched polymers. In some embodiments, polymers can be dendrimers. Polymers may be homopolymers or copolymers including two or more monomers. In terms of sequence, copolymers may be block copolymers, graft copolymers, random copolymers, blends, mixtures, and/or adducts of any of the foregoing and other polymers.

In some embodiments, particles may be made of or include a natural polymer, such as a carbohydrate, protein, nucleic acid, lipid, etc. In some embodiments, natural polymers may be synthetically manufactured. Many natural polymers, such as collagen, hyaluronic acid (HA), and fibrin, which derived from various components of the mammalian extracellular matrix can be used in particles. Collagen is one of the main proteins of the mammalian extracellular matrix, while HA is a polysaccharide that is found in nearly all animal tissues. Alginate and agarose are polysaccharides that are derived from marine algae sources. Some advantages of natural polymers include low toxicity and high biocompatibility.

In some embodiments, a polymer is a carbohydrate. In some embodiments, a carbohydrate may be a monosaccharide (i.e. simple sugar). In some embodiments, a carbohydrate may be a disaccharide, oligosaccharide, and/or polysaccharide including monosaccharides and/or their derivatives connected by glycosidic bonds. Although carbohydrates that are of use in classification and quantification of multifunctional objects are typically natural carbohydrates, they may be at least partially-synthetic. In some embodiments, a carbohydrate is a derivatized natural carbohydrate.

In certain embodiments, a carbohydrate is or includes a monosaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is or includes a disaccharide, including but not limited to lactose, sucrose, maltose, trehalose, and cellobiose. In certain embodiments, a carbohydrate is or includes a polysaccharide, including but not limited to hyaluronic acid (HA), alginate, heparin, agarose, chitosan, N,O-carboxylmethylchitosan, chitin, cellulose, microcrystalline cellulose, hydroxypropyl methylcellulose (HPMC), hydroxycellulose (HC), methylcellulose (MC), pullulan, dextran, cyclodextran, glycogen, starch, hydroxyethylstarch, carageenan, glycon, amylose, starch, heparin, konjac, glucommannan, pustulan, curdlan, and xanthan. In certain embodiments, the carbohydrate is a sugar alcohol, including but not limited to mannitol, sorbitol, xylitol, erythritol, maltitol, and lactitol.

In some embodiments, particles may be made of or include synthetic polymers, including, but not limited to, poly(arylates), poly(anhydrides), poly(hydroxy acids), poly(alkylene oxides), polypropylene fumerates), polymethacrylates polyacetals, polyethylenes, polycarbonates (e.g. poly(1,3-dioxan-2-one)), polyanhydrides (e.g. poly(sebacic anhydride)), polyhydroxyacids (e.g. poly(β-hydroxyalkanoate)), polypropylfumarates, polycaprolactones, polyamides (e.g. polycaprolactam), polyacetals, polyethers, polyesters (e.g. polylactide, polyglycolide, poly(dioxanones), polyhydroxybutyrate,), poly(orthoesters), polycyanoacrylates, polyvinyl alcohols, polyurethanes, polyphosphazenes, polyacrylates, polymethacrylates, polyureas, polyamines and copolymers thereof. Exemplary polymers also include polyvalerolactone, poly(sebacic anhydride), polyethylene glycol, polystyrenes, polyhydroxyvalyrate, poly(vinyl pyrrolidone) poly(hydroxyethyl methacrylate) (PHEMA), poly(vinyl alcohol) (PVA), and derivatives and copolymers thereof.

In some embodiments, photocrosslinking methods are utilized to make polymeric particles. Photoinitiators produce reactive free radical species that initiate the crosslinking and/or polymerization of monomers upon exposure to light. Any photoinitiator may be used in the crosslinking and/or polymerization reaction. Photoinitiated polymerizations and photoinitiators are discussed in detail in Rabek, Mechanisms of Photophysical Processes and Photochemical Reactions in Polymers, New York: Wiley & Sons, 1987; Fouassier, Photoinitiation, Photopolymerization, and Photocuring, Cincinnati, Ohio: Hanser/Gardner; Fisher et al., 2001, Annu Rev. Mater. Res., 31:171. A photoinitiator may be designed to produce free radicals at any wavelength of light. In certain embodiments, the photoinitiator is designed to work using UV light (200-500 nm). In certain embodiments, long UV rays are used. In other embodiments, short UV rays are used. In some embodiments, a photoinitiator is designed to work using visible light (400-800 nm). In certain embodiments, a photoinitiator is designed to work using blue light (420-500 nm). In some embodiments, the photinitiator is designed to work using IR light (800-2500 nm). The output of light can be controlled to provide greater control over the crosslinking and/or polymerization reaction. Control over polymerization in turn results in control over characteristics and/or properties of the resulting hydrogel.

In some embodiments, a particle can be or include an inorganic polymer such as silica (SiO2). In some embodiments, particles are silica-based. For example, silicate materials may be useful for the present applications due to their biocompatibility, ease of production and functionalization, and large surface-to-volume ratio. Silica-based particles such as porous silica particles, and any modified or hybrid particles can be of use in accordance with some embodiments of the present disclosure.

Silica-based particles may be made by a variety of methods. Some methods utilize the Stöber synthesis which involves hydrolysis of tetraethoxyorthosilicate (TEOS) catalyzed by ammonia in water/ethanol mixtures, or variations thereof. In some embodiments, silica-based particles are synthesized using known sol-gel chemistry, e.g., by hydrolysis of a silica precursor or precursors. Silica precursors can be provided as a solution of a silica precursor and/or a silica precursor derivative. Hydrolysis can be carried out under alkaline (basic) or acidic conditions. For example, hydrolysis can be carried out by addition of ammonium hydroxide to a solution including one or more silica precursor and/or derivatives. Silica precursors are compounds which under hydrolysis conditions can form silica. Examples of silica precursors include, but are not limited to, organosilanes such as, for example, tetraethoxysilane (TEOS), tetramethoxysilane (TMOS) and the like. In some embodiments, silica precursor has a functional group. Examples of such silica precursors includes, but is not limited to, isocyanatopropyltriethoxysilane (ICPTS), aminopropyltrimethoxysilane (APTS), mercaptopropyltrimethoxysilane (MPTS), and the like. In some embodiments, microemulsion procedures can be used to synthesize particles. For example, a water-in-oil emulsion in which water droplets are dispersed as nanosized liquid entities in a continuous domain of oil and surfactants and serve as nanoreactors for nanoparticle synthesis offer a convenient approach.

In some embodiments, particles may contain detectable moieties that generate fluorescent, luminescent and/or scatter signal. In certain embodiments, particles contain quantum dots (QDs). QDs are bright, fluorescent nanocrystals with physical dimensions small enough such that the effect of quantum confinement gives rise to unique optical and electronic properties. Semiconductor QDs are often composed of atoms from groups II-VI or III-V in the periodic table, but other compositions are possible. By varying their size and composition, the emission wavelength can be tuned (i.e., adjusted in a predictable and controllable manner) from the blue to the near infrared. QDs generally have a broad absorption spectrum and a narrow emission spectrum. Thus different QDs having distinguishable optical properties (e.g., peak emission wavelength) can be excited using a single source. In general, QDs are brighter and photostable than most conventional fluorescent dyes. QDs and methods for their synthesis are well known in the art (see, e.g., U.S. Pat. Nos. 6,322,901; 6,576,291; and 6,815,064; all of which are incorporated herein by reference). QDs can be rendered water soluble by applying coating layers including a variety of different materials (see, e.g., U.S. Pat. Nos. 6,423,551; 6,251,303; 6,319,426; 6,426,513; 6,444,143; and 6,649,138; all of which are incorporated herein by reference). For example, QDs can be solubilized using amphiphilic polymers. Exemplary polymers that have been employed include octylamine-modified low molecular weight polyacrylic acid, polyethylene-glycol (PEG)-derivatized phospholipids, polyanhydrides, block copolymers, etc.

Exemplary QDs suitable for use in some embodiments, includes ones with a wide variety of absorption and emission spectra and they are commercially available, e.g., from Quantum Dot Corp. (Hayward Calif.; now owned by Invitrogen) or from Evident Technologies (Troy, N.Y.). For example, QDs having peak emission wavelengths of approximately 525 nm, approximately 535 nm, approximately 545 nm, approximately 565 nm, approximately 585 nm, approximately 605 nm, approximately 655 nm, approximately 705 nm, and approximately 800 nm are available. Thus QDs can have a range of different colors across the visible portion of the spectrum and in some cases even beyond.

In certain embodiments, optically detectable particles are or include metal particles. Metals of use include, but are not limited to, gold, silver, iron, cobalt, zinc, cadmium, nickel, gadolinium, chromium, copper, manganese, palladium, tin, and alloys thereof. Oxides of any of these metals can be used.

Certain metal particles, referred to as plasmon resonant particles, exhibit the well known phenomenon of plasmon resonance. The features of the spectrum of a plasmon resonant particle (e.g., peak wavelength) depend on a number of factors, including the particle's material composition, the shape and size of the particle, the refractive index or dielectric properties of the surrounding medium, and the presence of other particles in the vicinity. Selection of particular particle shapes, sizes, and compositions makes it possible to produce particles with a wide range of distinguishable optically detectable properties thus allowing for concurrent detection of multiple analytes by using particles with different properties such as peak scattering wavelength.

Magnetic properties of particles can be used in classification and quantification of multifunctional objects. Particles in some embodiments are or include magnetic particles, that is, magnetically responsive particles that contain one or more metals or oxides or hydroxides thereof. Magnetic particles may include one or more ferrimagnetic, ferromagnetic, paramagnetic, and/or superparamagnetic materials. Useful particles may be made entirely or in part of one or more materials selected from the group consisting of: iron, cobalt, nickel, niobium, magnetic iron oxides, hydroxides such as maghemite (γ-Fe2O3), magnetite (Fe3O4), feroxyhyte (FeO(OH)), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the afore-mentioned oxides or hydroxides, and mixtures of any of the foregoing. See, e.g., U.S. Pat. No. 5,916,539 (incorporated herein by reference) for suitable synthesis methods for certain of these particles. Additional materials that may be used in magnetic particles include yttrium, europium, and vanadium.

In general, particles suitable for classification and quantification of multifunctional objects can be of any size. In some embodiments, suitable particles have a greatest dimension (e.g. diameter) of less than 1000 micrometers (μm). In some embodiments, suitable particles have a greatest dimension of less than 500 μm. In some embodiments, suitable particles have a greatest dimension of less than about 250 μm. In some embodiments, suitable particles have a greatest dimension (e.g. diameter) of less than about 200 μm, about 150 μm, about 100 μm, about 90 μm, about 80 μm, about 70 μm, about 60 μm, about 50 μm, about 40 μm, about 30 μm, about 20 μm, or about 10 μm. In some embodiments, suitable particles have a greatest dimension of less than 1000 nm. In some embodiments, suitable particles have a greatest dimension of less than 500 nm. In some embodiments, suitable particles have a greatest dimension of less than about 250 nm. In some embodiments, a greatest dimension is a hydrodynamic diameter.

Suitable particles can have a variety of different shapes including, but not limited to, spheres, oblate spheroids, cylinders, ovals, ellipses, shells, cubes, cuboids, cones, pyramids, rods (e.g., cylinders or elongated structures having a square or rectangular cross-section), tetrapods (particles having four leg-like appendages), triangles, prisms, etc. In some embodiments, particles are rod-shaped. In some embodiments, particles are bar-shaped. In some embodiments, particles are bead-shaped. In some embodiments, particles are column-shaped. In some embodiments, particles are ribbon or chain-like. In some embodiments, particles can be of any geometry or symmetry. For example, planar, circular, rounded, tubular, ring-shaped, tetrahedral, hexagonal, octagonal particles, particles of other regular geometries, and/or particles of irregular geometries can also be used in classification and quantification of multifunctional objects. Additional suitable particles with various sizes and shapes are disclosed in U.S. Pat. No. 7,709,544 and U.S. Pat. No. 7,947,487, which are incorporated herein by reference.

Particles may have various aspect ratios of their dimensions, such as length/width, length/thickness, etc. Particles, in some embodiments, can have at least one dimension, such as length, that is longer than another dimension, such as width. According to some embodiments, particles having at least one aspect ratio greater than one may be particularly useful in flow-through scanning (e.g., in a flow cytometer) to facilitate their self-alignment. In some embodiments, particles may have at least one aspect ratio of at least 1.5:1, at least 2:1, at least 2.5:1, at least 3:1, at least 5:1, at least 10:1, at least 15:1, or even greater.

It is often desirable to use a population of particles that is relatively uniform in terms of size, shape, and/or composition so that each particle has similar properties. In some embodiments, a population of particles with homogeneity with diameters (e.g., hydrodynamic diameters) are used. As used herein, a population of particles with homogeneity with diameters (e.g., hydrodynamic diameters) refers to a population of particles with at least about 80%, at least about 90%, or at least about 95% of particles with a diameter (e.g., hydrodynamic diameter) that falls within 5%, 10%, or 20% of the average diameter (e.g., hydrodynamic diameter). In some embodiments, the average diameter (e.g., hydrodynamic diameter) of a population of particles with homogeneity with diameters (e.g., hydrodynamic diameters) ranges as discussed above. In some embodiments, a population of particles with homogeneity with diameters (e.g., hydrodynamic diameters) refers to a population of particles that has a polydispersity index less than 0.2, 0.1, 0.05, 0.01, or 0.005. For example, polydispersity index of particles is in a range of about 0.005 to about 0.1. Without wishing to be bound by any theory, it is contemplated that particles with homogeneity (e.g., with respect to particle size) may have higher repeatability and can produce more accuracy in the present application. In some embodiments, a population of particles may be heterogeneous with respect to size, shape, and/or composition.

Particles can be solid or hollow and can include one or more layers (e.g., nanoshells, nanorings, etc.). Particles may have a core/shell structure, wherein the core(s) and shell(s) can be made of different materials. Particles may include gradient or homogeneous alloys. Particles may be composite particles made of two or more materials, of which one, more than one, or all of the materials possesses magnetic properties, electrically detectable properties, and/or optically detectable properties.

Particles may have a coating layer. Use of a biocompatible coating layer can be advantageous, e.g., if the particles contain materials that are toxic to cells. Suitable coating materials include, but are not limited to, natural proteins such as bovine serum albumin (BSA), biocompatible hydrophilic polymers such as polyethylene glycol (PEG) or a PEG derivative, phospholipid-(PEG), silica, lipids, polymers, carbohydrates such as dextran, other nanoparticles that can be associated with inventive nanoparticles etc. Coatings may be applied or assembled in a variety of ways such as by dipping, using a layer-by-layer technique, by self-assembly, conjugation, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties. In some embodiments, particles with coating are also referred to as functionalized particles or surface treated particles.

In certain embodiments, a particle is porous, by which is meant that the particle contains holes or channels, which are typically small compared with the size of a particle. For example a particle may be a porous silica particle, e.g., a porous silica nanoparticle or may have a coating of porous silica. Particles may have pores ranging from about 1 nm to about 200 nm in diameter, e.g., between about 1 nm and 50 nm in diameter. Between about 10% and 95% of the volume of a particle may consist of voids within the pores or channels.

In some embodiments, particles may include one or more dispersion media, surfactants, release-retarding ingredients, or other pharmaceutically acceptable excipient. In some embodiments, particles may include one or more plasticizers or additives.

In various embodiments, particles described herein may have at least one region bearing one or more probes described herein. In some embodiments, particles may have at least one encoded region. In some embodiments, particles have at least one encoded region and at least one region bearing one or more probes. Such regions can be discrete regions of substrates (objects) including particles. Each region, in some embodiments, can be optionally functionalized. In various embodiments, particles described herein may bear an indicator for orientation (e.g., indicating coding region first followed by probe region or vice versa).

### Data Acquisition

Commercial flow cytometers can write data for each detected event in a standard flow cytometry data file, formatted according to the FCS standard. In some embodiments, software algorithms may contain methods to parse such files and extract an array of data, with one numerical value for each event and channel combination.

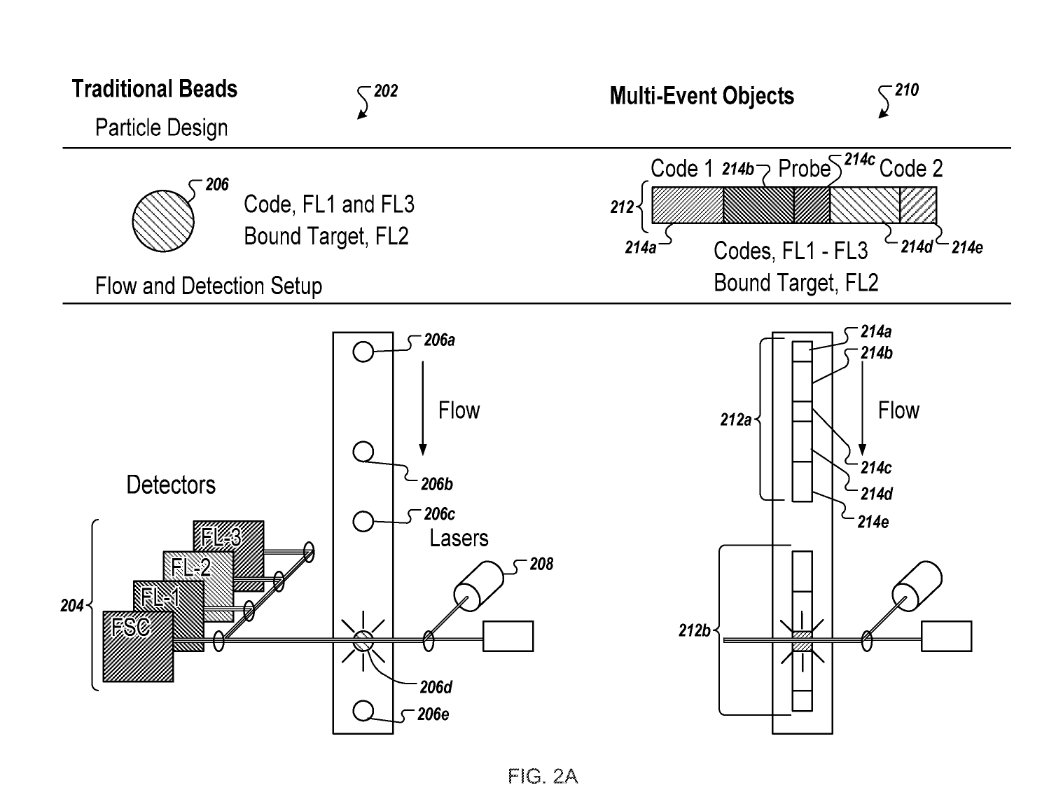

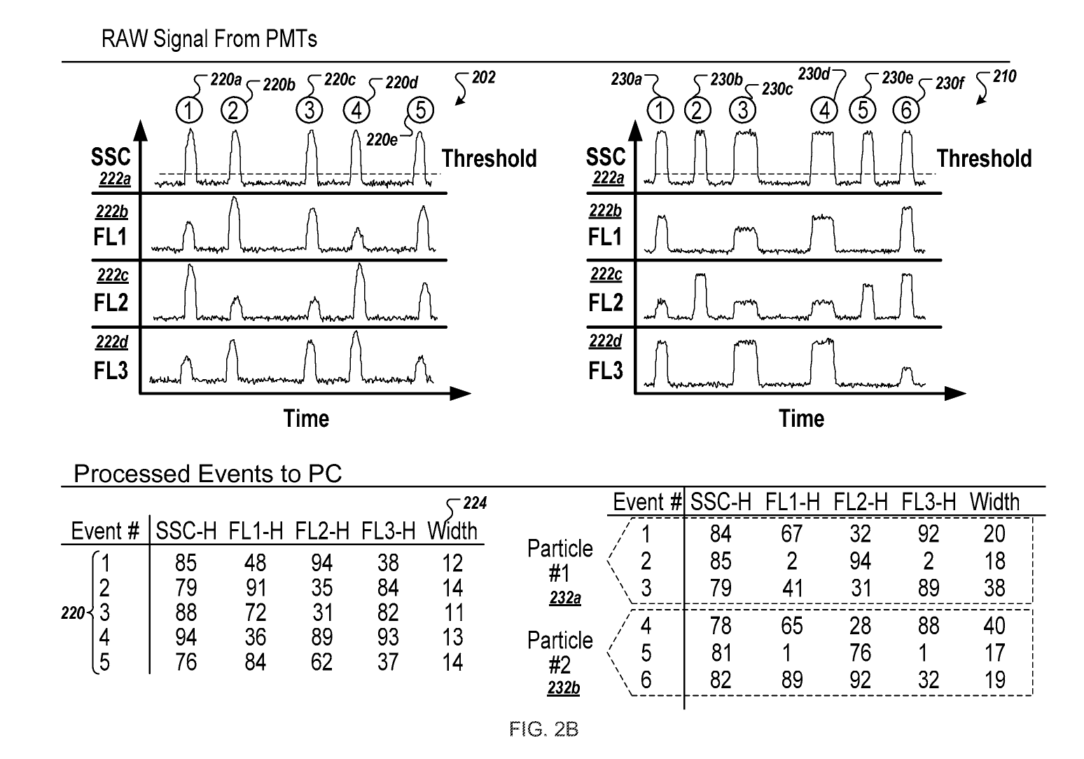

Turning to FIG. 2A, as illustrated in a left-hand column 202, standard cytometers record “events” as instances where the signal from a selected detector 204 breaks a threshold, recording single beads 206 as single events, and saving data for each channel. In some implementations, the signal may be caused by external excitation, such as by one or more lasers 208.

Moving to a right-hand column 210, in comparison, multifunctional particles 212, in some implementations, bear functional regions 214 that can be doped with triggering entities (that cause scatter for instance) and single particles 212 are recorded as multiple events by detectors (not illustrated).

### Object Recognition

In some embodiments, recognition of multifunctional objects begins with the grouping of events such that each group can be clearly identified as originating from the same physical object. There are multiple ways to do so, each with limitations depending on the recognition hardware (e.g., cytometer capabilities, etc.). In some implementations, several different methods of grouping events may be used and the results of the grouping combined in analysis, for example to enable an accurate determination of which events constitute an object. To allow such combination, in some implementations, each method may be used to assign a fit function F to a candidate sequence of events. For example, F=0 if the events are fully consistent with coming from the same object, and F>0 to the extent that this is not the case. In some implementations, the fit functions from multiple methods may be combined in a weighted sum to arrive at a consensus fit function. In the following paragraphs, examples of various fit functions are described.

Turning to FIG. 2B, as illustrated in a first column 202, the output of identification of beads via a flow cytometer can be illustrated as a series of signals 220 (e.g., events) captured over time. Each event, for example, may be associated with one or more signal detections 222 (e.g., SSC, FL1, FL2, FL3) as well as signal size and/or durations (e.g., timestamp, width 224, etc.).

Moving to the second column 210 of FIG. 2B, a second series of signals 230, captured in relation to multifunctional objects 212, may be grouped into a series of objects (e.g., particles) 232, where each of the particles are identified as including three separate events.

If sufficient resolution is available in the TIME channel, in some implementations, groups of events can be identified by their time difference. For example, sufficient resolution may be determined based in part upon an estimated distance between regions of an object (e.g., approximately 10 to 15 micrometers apart). In some implementations, sufficient resolution may be determined based in part upon an approximate flow speed of the particle analyzer (e.g., cytometer). For example, the flow speed of a typical cytometer may range from 0.1 to 10 meters per second. In some examples, a sufficient time resolution may be less than 100 microseconds, about ten microseconds, or about one microsecond. An object may be characterized by a sequence of Nr events following each other at well-defined time intervals, where Nr refers to the number of regions per object. The interval between objects, for example, may typically be much larger than the interval between regions of the same object. In some implementations, the interval between objects may vary widely. Given a candidate set of Nr regions, in some implementations, an algorithm may be used to determine the Nr−1 successive time intervals dTi (i=1 . . . Nr−1), and calculate a set of partial fit functions Fi as follows. If dTi is within the interval ranging from dT−tol to dT+tol, where tol refers to the tolerance of time interval (e.g., range of time where the fit function F may fall within a perfect fit range of F=0), Fi is zero. Otherwise, Fi equals the square of the amount that dTi is outside of this interval. The total object fit function F may then be calculated as the sum of the Nr−1 partial fit functions. The values of dT and tol, in some implementations, may be optimized according to the type of cytometer and/or a particle dimension (e.g., distance between regions, total length of particle, etc.). In some implementations, the distance between regions divided by dT may be approximately equal to the flow velocity, and tol may be a fraction of dT, such as 20%.

In some implementations, objects may be recognized in part by patterns of successive fluorescence intensities. Patterns may consist of pre-defined levels of fluorescence in the active regions of the objects in one or multiple detection channels, for example in the trigger channel. Objects, in some implementations, may be prepared in such a way that the intensity in one channel differs across the regions in a known pattern of intensities, for example Pi (j=1 . . . Nr). The pattern may be normalized such that ΣPi=1. A given set of intensities from a candidate sequence of events in the pattern channel may be similarly normalized to yield Ii (i=1 . . . Nr), ΣIi=1. The fit function may be obtained as the root-mean-square (RMS) difference: F=Σ(Ii−Pi)2. More than one channel could be used this way, in some implementations, with all fit functions combined in a weighted sum or other suitable combination.

Most cytometers record the duration of a fluorescence signal in addition to its intensity, often reported as three different properties of the observed signal peak: height, width, and area. Only two of these are generally independent measurements, the third is often calculated. Area is typically the most representative of the amount of fluorescence present. By varying the physical size of the regions, in some implementations, width can be used as an additional variable to help identify and orient objects. In some embodiments, width may be used optionally to increase the accuracy of orientation, for example using a qualitative comparison between the widths of the two code regions. In some implementations, a fit function relative to width may be defined similar to the algorithms presented in relation to time and intensities, for example as described above.

Objects may typically be physically oriented in the flow such that they transit the analysis region of the flow cell lengthwise. However, the order of regions may be forward (e.g., region 1 first) or reverse (e.g., region Nr first), with equal probability. Objects, in some implementations, may be designed asymmetrically. To detect asymmetrically-designed particles, in some implementations, one or more algorithms may be used to uniquely determine orientation. This can be achieved, for example, by using asymmetric patterns of region distance (for time detection), intensity, and/or width. Determination of orientation, in some implementations, may be achieved by calculating the fit function F both ways and selecting the best match. In some embodiments, orientation may be encoded as an intensity difference in the trigger channel.

### Object Identification

Once it is known which events compose an object, each event can be uniquely assigned to an object region. In some embodiments, two code regions may be used to identify each object using discrete levels of fluorescence intensities in one channel, and one probe region may be used to read out the assay. Other combinations, with more or less code regions and multiple probe regions are also possible. Multiple probe regions, in particular, would allow the read-out of multiple assays on a single particles, which could be advantageous as particle-particle variation would be eliminated as a source of error in differential measurements.

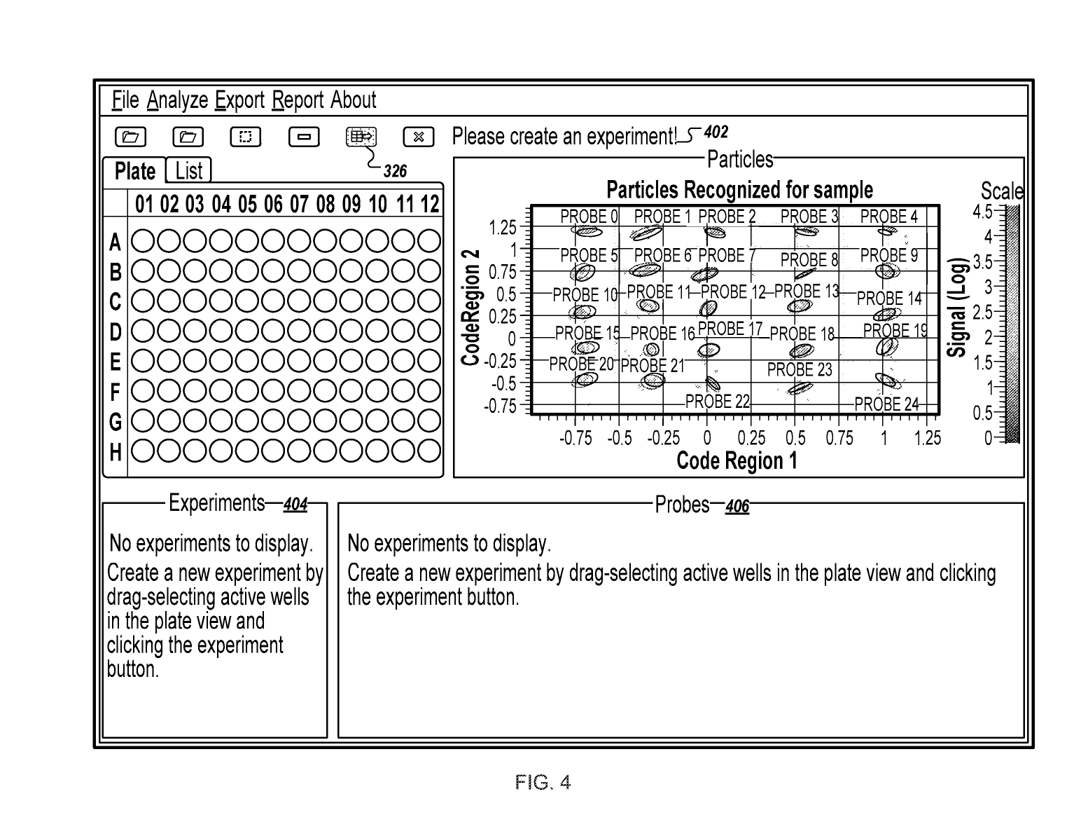

Raw levels of detected fluorescence may vary from object to object for various reasons. To get accurate measurements, it may be advantageous to compute ratios between two channels, which are much less variable. For example, as illustrated in a particle view 408 of FIG. 4, green-normalized yellow code levels are used for recognition of twenty-five different classes of particle. The logarithmic yellow/green (Y/G) ratios for the two different code regions are plotted for each particle, with clusters outlined by ellipses indicating the fitted bivariate normal distribution for each code. Particles are colored according to the logarithmic Y/G signal in the probe region.

In some embodiments, particles may be functionalized with a green fluorophore in all three regions for trigger, and a yellow fluorophore for code and assay signals. The trigger (green) may be identical across all particles, but the signal (yellow) may vary according to code and assay response. In some embodiments, the detector used for triggering may also be used for normalization. Accurate quantitative signals for each region may be derived by dividing the yellow intensity by the green intensity. With five distinct levels of yellow intensity in two code regions, 25 different types of particles can be distinguished, for example as shown in FIG. 4.

With typical cytometers having at least three channels, and three regions per particle, the total number of independent variables available for coding is eight, assuming one variable is used for assay readout. This illustrates the great advantage in coding capability that multi-functional particles provide over bead based methods. In some embodiments, the probe region may contain only the trigger and the assay signal, with no variation between particles, for example in order to minimize background and cross-talk between code and signal. In addition, all signals may be normalized as described above. This leaves 2 variables in each code region for identification. With 5 levels in each variable, 54=625 particle identifiers are possible. With 8 levels, the number of possible combinations is 4096. In some embodiments, only two channels are used to minimize requirements on the cytometer. This permits the use of simple devices, for example with only a single excitation laser and two fluorescence detectors.

### Signal Quantification

Signal quantification includes selecting particularly advantageous combinations (e.g., ratio, logarithm, etc.) of measured quantities from each object. The selected signal measurements, in turn, may be statistically integrated over multiple objects (e.g., identified as belonging a type having the same code or otherwise representing a same test group such as the same assay). Statistical integration, for example, may be achieved using one or more methods such as means and quantiles to obtain an accurate estimate of the true sample property that the test (e.g., assay) is designed to determine. Signal quantification, in a particular use, refers to the process of measuring the amount of signal generated by the actual assay on the probe region of the object. In some embodiments, a yellow fluorescent conjugate may be used for the assay, preferably streptavidin phycoerythrin (SAPE).

If the green channel is used as the trigger, it can be used for normalization as well, and the assay readout can be accurately measured as Y/G as is done for the code levels. Alternatively, to reduce potential interference caused by extra fluorescence in the probe region, a combination of intensities of the code regions can be used to normalize the probe signal.

In cases of very high assay signal, a fluorescence channel may become saturated, i.e. the incoming light may exceed the range of the detector. Normally, this would limit the dynamic range of the measurement. In some implementations, the normally undesirable spectral overlap between adjacent channels may be used to accurately measure the assay response even when the primary channel is saturated. This is particularly advantageous when there exists a spectrally adjacent channel that is not used for other purposes. The signal in this channel will then be proportional to assay response, but only at a small fraction of the primary channel intensity, thereby escaping saturation. This fraction, known in the art as bleed-through, can be known a priori, or it can be inferred on the fly from measuring events that are close to, but not yet in saturation. Dividing the signal in the secondary channel by the bleed-through fraction yields the primary channel signal expected without saturation. In some implementations, red may be used for triggering all regions and normalizing the probe signal. Further to the example, green may be used for code normalization and probe dynamic range, and yellow for code and probe signal. More than one additional channel could be used for dynamic range, which would extend such range even further.

To obtain a reliable single reporter value for each assay, in some implementations, the probe signals of all objects with the same code may be integrated. This is advantageous, as it increases the measurement accuracy by reducing the standard error of the combined signal.

There are many techniques to do this such as, in some examples, a) the mean (arithmetic, geometric or harmonic), b) the median, c) the midpoint of the range, etc. In some embodiments, the signal values of all objects with the same code may first be ordered by numerical value. In some examples, the top and bottom 5, 10, 25 or 40 (25 is used in the preferred embodiment) percent may be disregarded as outliers. In a particular embodiment, the top and bottom 25 percent of the signal values of all objects may be discarded. After having discarded a percentage of outlier signal values, a Gaussian statistic may be calculated for the remaining signal values, yielding a mean and confidence interval of measurement. To avoid under-estimation of the error, in some implementations, a correction factor may be applied to account for the non-normality of the truncated normal distribution.

### User Interface

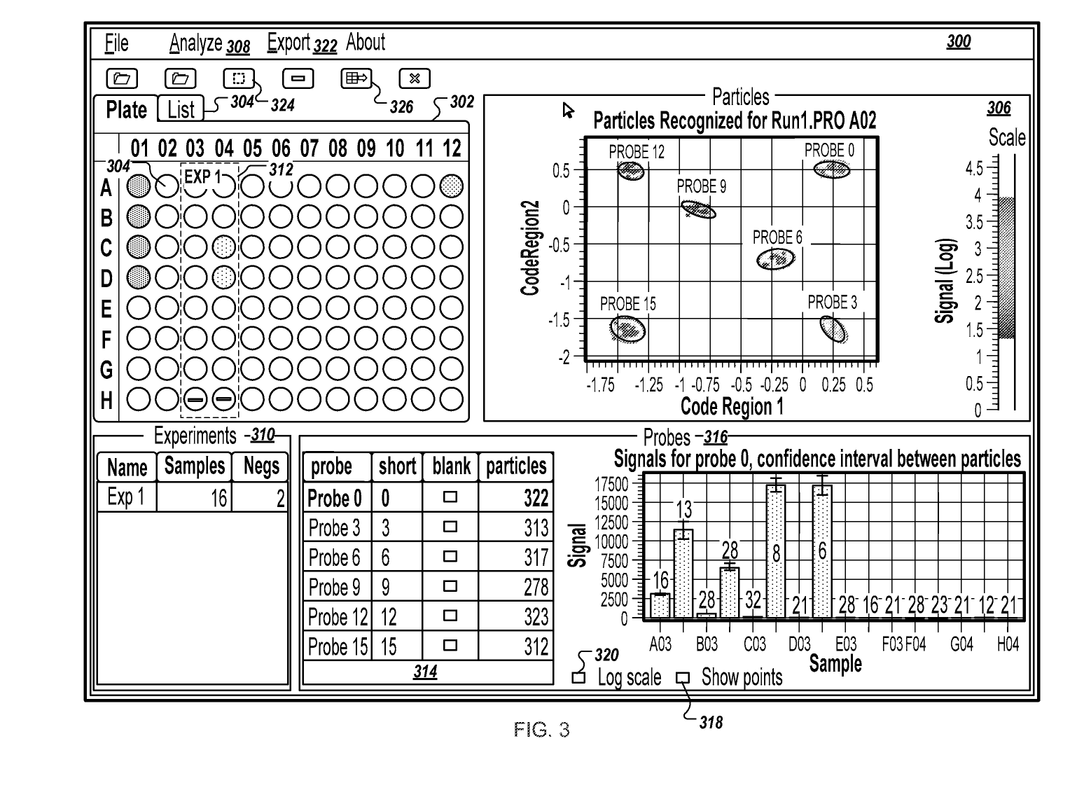

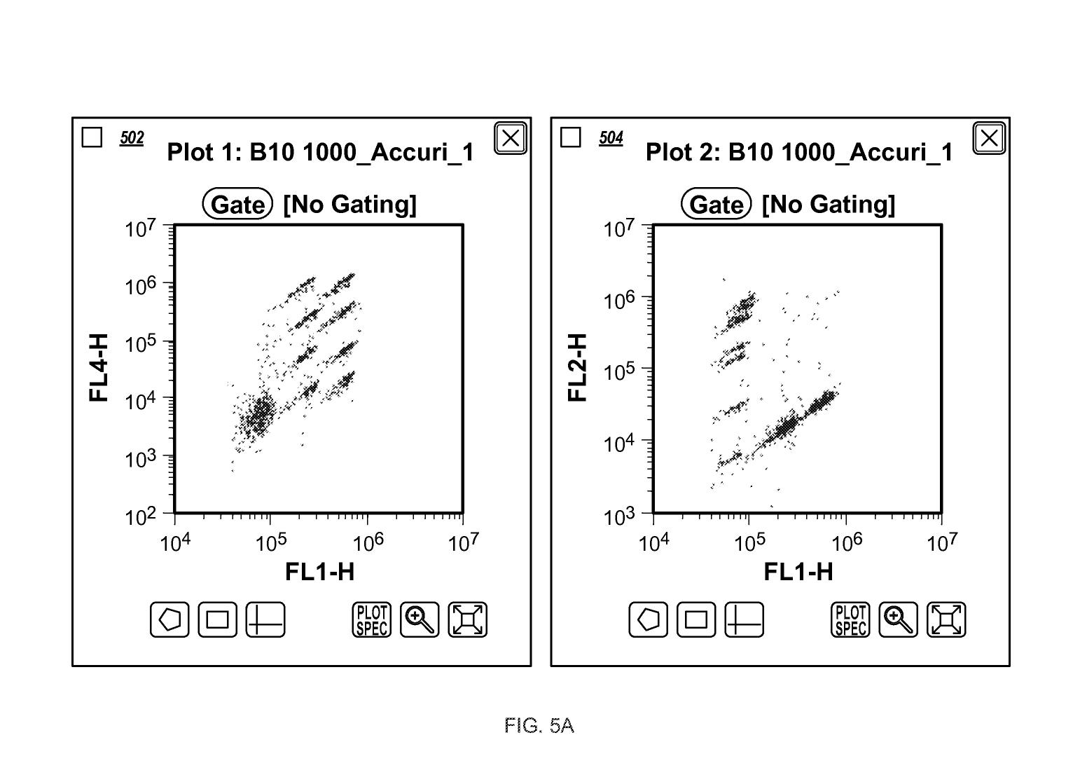

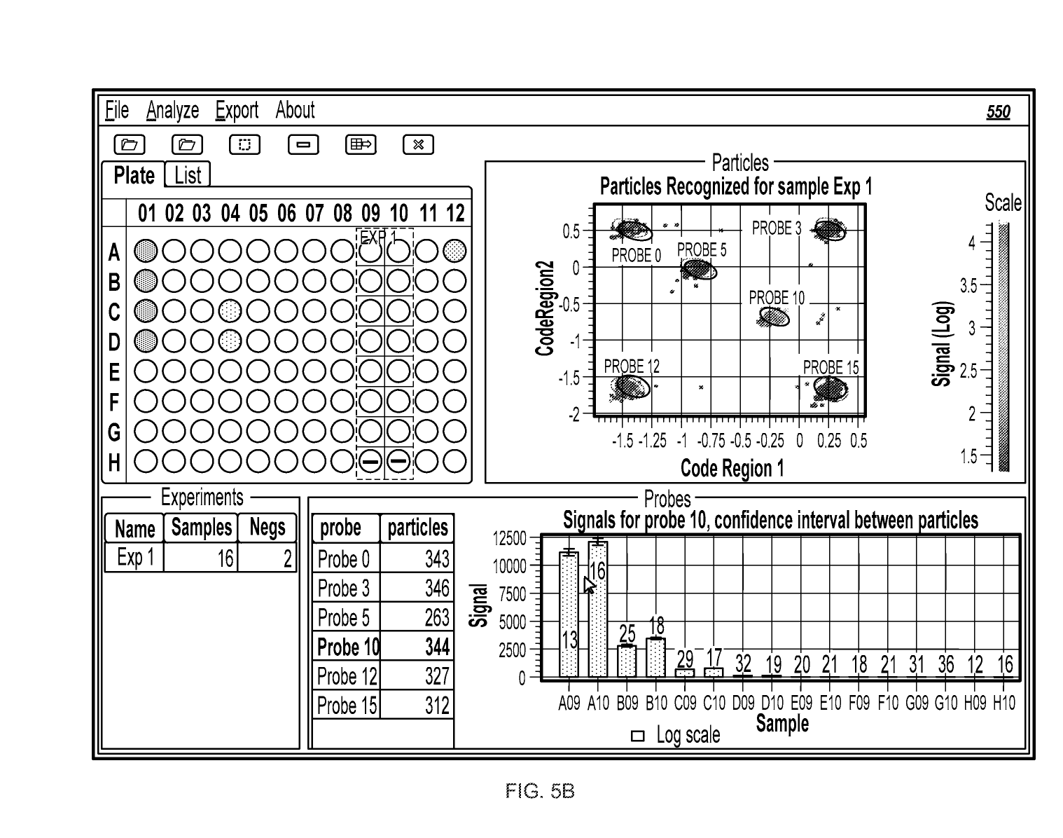

Turning to FIG. 3, in some implementations, a user interface 300 may be designed to minimize the amount of action required by the user in order to accomplish common tasks. The major steps involved in data analysis and supported by the program are as follows: (a) load a raw data file, such as a flow cytometry file (FCS-file), (b) select samples to define an experiment, (c) view the data, and (d) export the data. For example, as illustrated in relation to FIG. 5A, raw data illustrated in plots 502 and 504, may be collected by particle detection apparatus and provided to analysis software. Turning to FIG. 5B, the analysis software may present a user interface 550 for selecting samples, reviewing and analyzing the object information identified by the analysis software. Upon review and configuration (described in greater detail in relation to FIGS. 3 and 4), a user may export the data.

As the FCS record for each sample is loaded, the software may determine the plate location of the corresponding sample. As illustrated in FIG. 3, plate locations may be visibly represented by a plate array 302. Most preferably, the location may be extracted from tags contained in the FCS file. Alternatively plate locations can be parsed from the sample name by detecting subsequences of the form ‘A01’, where ‘A’ could be any plate row (usually A-H) and ‘01’ any plate column (usually 1-12).

As the data are processed according to the methods described above, in some implementations, a quality factor Q may be determined as the product of the fraction of events recognized and the fraction of particles assigned to code clusters. The factor Q, in some examples, may range from 0 to 1, and two thresholds may be applied to classify samples as good, questionable, and bad. These are indications, for example, of how likely the result of the analysis is to be correct. The factor Q, for example, may approximately equate to the portion of events that information can be successfully extracted from, taking into account both recognition and identification. Preferred thresholds, for example, may be Q>80% for good, 80%>Q>50% for questionable, and Q<50% for bad. As illustrated in FIG. 3, the plate wells may be colored to indicate the QC classifications calculated for each sample. Preferred colors, for example, may be green for good, yellow for questionable, and red for bad.

In addition to the plate layout, in some implementations, the plate view can be switched to display a table of samples using the “List” tab 304 at the top of the view. This is particularly useful if plate location information is not available for some or all samples. Samples may be designated negative controls using the checkboxes in the sample table. In the plate view, negative controls may be indicated with a minus sign.

When a sample well is selected (e.g., well A02 304), the code clusters for this sample are displayed in a code view 306. The code view 306 includes a plot of the normalized code signal of region 1 on the x-axis and region 2 on the y-axis. Each particle detected in the sample is displayed as a colored dot. The color, in some implementations, corresponds to the normalized signal on the probe region, and is quantified by the color scale to the right of the plot. Code clusters that the software was able to identify, in some implementations, may be delineated with an ellipse that follows the contours of the bivariate normal distribution of the cluster. The plot can be zoomed and panned using the mouse and keyboard. Detailed information on each particle can be obtained by hovering the mouse pointer over it. Particles can be selected individually or in groups by clicking or dragging the mouse.

Before analysis, the user may select a subset of samples using the mouse or keyboard in the plate view and create an experiment by clicking a “New experiment” button 324, by right-clicking and selecting “New experiment” from the menu, or by selecting “Replicates in rows” or “Replicates in columns” from an “Analyze” menu 308. In the latter case, samples are grouped into replicates as indicated, in the remaining cases samples are grouped into replicates along the shorter dimension of the selection. For example, if an area 3 columns wide and 8 rows high is selected and the “New Experiment” button clicked, an experiment will be defined with 8 samples assumed to be run in 3 replicates each. Any experiments so defined are displayed in a list 310 titled “Experiments” in the bottom left corner of the main screen. The list contains the name, number of samples, and number of negative controls for each defined experiment. Experiments are also indicated in the plate view 302 by surrounding its samples with a dashed boundary 312 labeled with the name of the experiment in the upper left corner. The user may edit the names of the experiments in the table. Experiments can be selected by clicking on a table row, and the currently selected experiment is indicated by coloring the row.

Next to the table of experiments is a table of probes 314. The table of probes 314 contains the name of the probe and the number of particles detected that carry it. Preferably, the probes are named after the biochemical analyte that they have been designed to detect. A column named “blank” with checkboxes is included to show the status of a probe as blank, i.e. there is no probe attached to particles with this code. The user may edit the names of the probes and the blank designations in the table. Probes can be selected by clicking on a table row, and the currently selected probe is indicated by coloring of the row.

If a probe is selected, the lower right corner of the main screen contains a bar graph 316 showing the measured signal for each probe. There is one bar for each sample. The amplitude of the signal is indicated by the height of the bar, and the confidence interval by error bars. In some embodiments, the individual particle measurements can also be indicated; this is controlled by the checkbox 318 located under the plot and labeled “Show points”. The scale of the y-axis is linear, unless a “Log scale” checkbox 320 is checked, in which case it becomes logarithmic. If negative controls have been selected, signals shown are background subtracted. If blank probes have been designated, their value will also have been subtracted. Blank probe subtraction is done before negative sample subtraction.

Before export, the software can be used to manipulate data for presentation. Data analysis may include subtraction of signal from “blank” particles from other particles within a well, subtraction of target signal in negative control wells from the other wells, or normalization using targets that include spike-in or endogenous species, or absolute target quantitation using calibration data. The software can also be used to present data across targets and samples in the format of heatmaps or other visual representations. Data may include particle-to-particle or well-to-well deviation, or signal-to-noise measurements.

If all parameters have been set as desired, the data can be exported into a tabular format suitable for further processing using spreadsheets or other data analysis software. This is achieved by clicking the “Export” button 326, or by selecting from an “Export” menu 322. The latter allows exporting the currently selected experiment, or all experiments together. In some examples, data may be exported into formats appropriate for statistics software such as SAS, SPSS, S+, or R, data analysis software such as MatLab, Mathematica, Spotfire, or electronic lab notebooks.

The progress of analysis, in some implementations, is tracked by the workflow control mechanism. There are two milestones in the workflow: “samples loaded” and “experiments defined.” The purpose of the flow is to reflect in the user interface which actions are required and which are not allowed. The buttons at the top of the screen are arranged in the order in which they should be used. As long as no samples have been loaded, no experiments can be defined or results exported and the corresponding buttons are shaded gray and cannot be activated. As samples are loaded and experiments defined, more of the buttons become available as appropriate. For example, as illustrated in FIG. 4, the Export Data button 326 is “grayed out” and unavailable for selection, while in FIG. 3, the Export Data button 326 is active (e.g., available for selection). A message to the right of the buttons reminds the user what action needs to be taken to get to the next milestone. For example, turning to FIG. 4, a message 402 encourages the user to “Please create an experiment!”. Any views on the main screen that do not yet have data to display show instead a short explanation of what action is required to obtain the missing information, as illustrated in an experiments view 404 and a probes view 406.

### Computing Network

In some implementations, a software application for recognition and analysis of multi-region particles may be installed upon a local computing device. The software application, for example, may be provided with the purchase of multi-region particles. In some implementations, a Web portal, for example accessible via a browser, may provide the user with the ability to access one or more recognition and analysis algorithms, such as the group identifier 114, the object identifier 116, and the report generator 118 described in relation to FIG. 1. For example, as discussed in relation to FIG. 1, a raw data file obtained from the flow cytometry system 104 may be uploaded to a network environment (e.g., via the network 102) for analysis, and results of the analysis may be presented to a user within a browser environment (e.g., upon the display 108). In some implementations, a portion of the software environment for recognition and analysis of multi-region particles may be executed upon a local computing device, while various algorithms may be performed by one or more remote processors, for example within a cloud computing environment. In some implementations, a user may download and execute software on a local computing device through a Web page using the Java™ Web Start framework by Oracle® of Santa Clara, Calif.

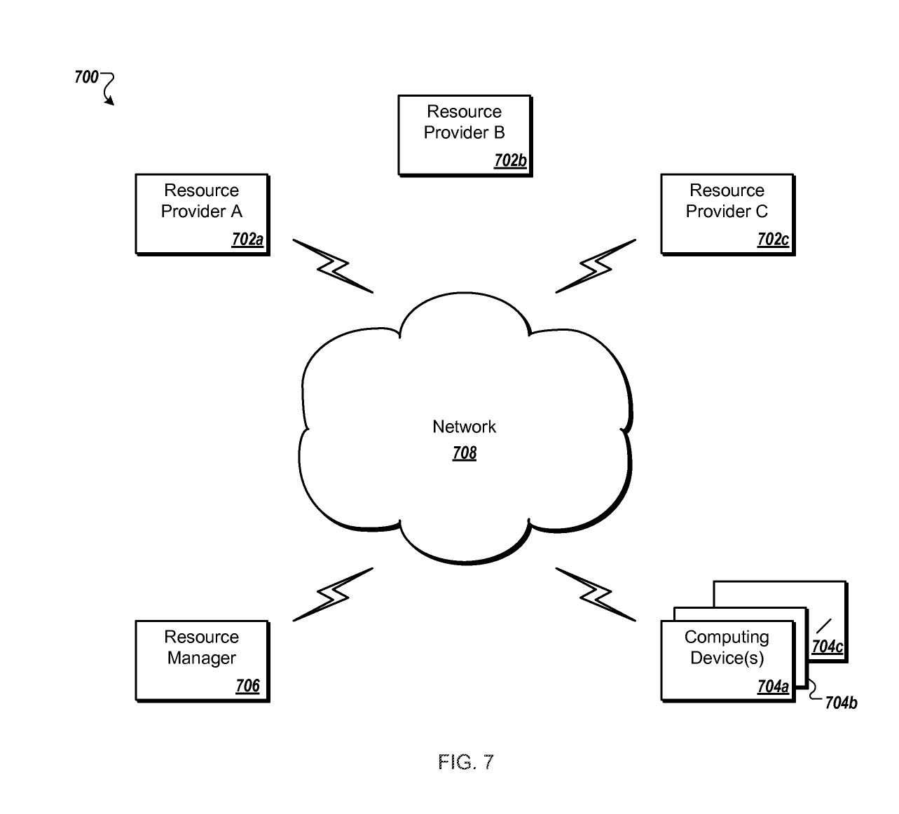

As shown in FIG. 7, an implementation of an exemplary cloud computing environment 700 for classification and quantification of multi-region particles is shown and described. In brief overview, the cloud computing environment 700 may include one or more resource providers 702a, 702b, 702c (collectively, 702). Each resource provider 702 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 702 may be connected to any other resource provider 702 in the cloud computing environment 700. In some implementations, the resource providers 702 may be connected over a computer network 708. Each resource provider 702 may be connected to one or more computing device 704a, 704b, 704c (collectively, 704), over the computer network 708.

The cloud computing environment 700 may include a resource manager 706. The resource manager 706 may be connected to the resource providers 702 and the computing devices 704 over the computer network 708. In some implementations, the resource manager 706 may facilitate the provision of computing resources by one or more resource providers 702 to one or more computing devices 704. The resource manager 706 may receive a request for a computing resource from a particular computing device 704. The resource manager 706 may identify one or more resource providers 702 capable of providing the computing resource requested by the computing device 704. The resource manager 706 may select a resource provider 702 to provide the computing resource. The resource manager 706 may facilitate a connection between the resource provider 702 and a particular computing device 704. In some implementations, the resource manager 706 may establish a connection between a particular resource provider 702 and a particular computing device 704. In some implementations, the resource manager 706 may redirect a particular computing device 704 to a particular resource provider 702 with the requested computing resource.

### Computing Devices

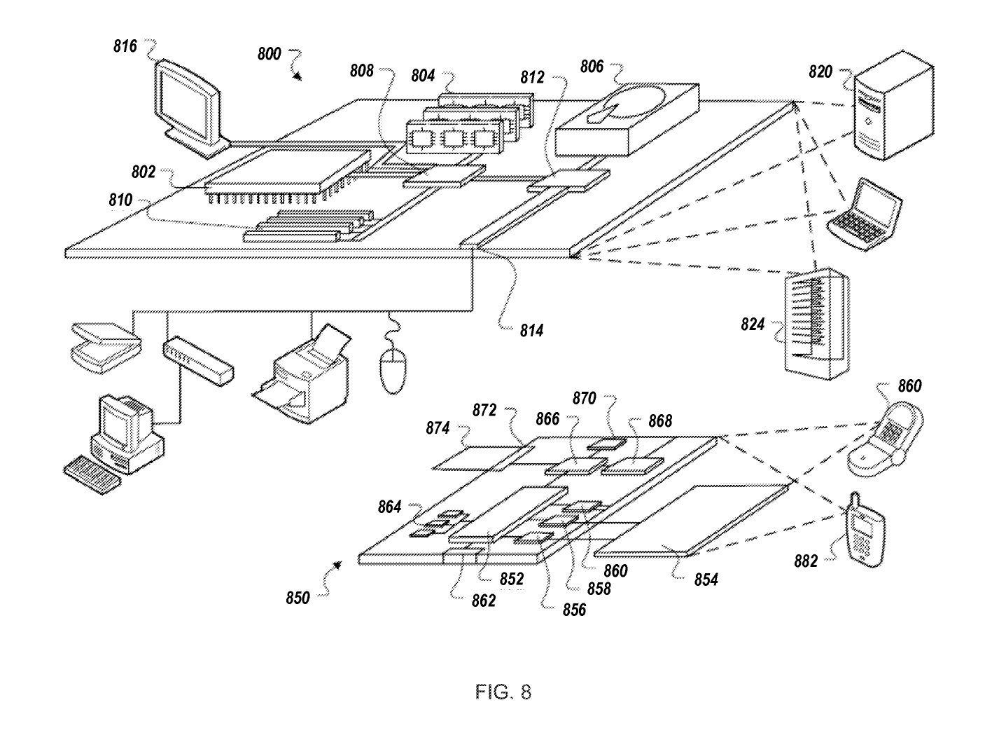

FIG. 8 shows an example of a computing device 800 and a mobile computing device 850 that can be used to implement the techniques described in this disclosure. The computing device 800 is intended to represent various forms of digital computers, such as laptops, desktops, workstations, personal digital assistants, servers, blade servers, mainframes, and other appropriate computers. The mobile computing device 850 is intended to represent various forms of mobile devices, such as personal digital assistants, cellular telephones, smart-phones, tablet computers, netbook computers, and other similar computing devices. The components shown here, their connections and relationships, and their functions, are meant to be examples only, and are not meant to be limiting.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and may be mounted on a common motherboard or in other manners as appropriate. The processor 802 can process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 804 stores information within the computing device 800. In some implementations, the memory 804 is a volatile memory unit or units. In some implementations, the memory 804 is a non-volatile memory unit or units. The memory 804 may also be another form of computer-readable medium, such as a magnetic or optical disk.

The storage device 806 is capable of providing mass storage for the computing device 800. In some implementations, the storage device 806 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 802), perform one or more methods, such as those described above. The instructions can also be stored by one or more storage devices such as computer- or machine-readable mediums (for example, the memory 804, the storage device 806, or memory on the processor 802).

The high-speed interface 808 manages bandwidth-intensive operations for the computing device 800, while the low-speed interface 812 manages lower bandwidth-intensive operations. Such allocation of functions is an example only. In some implementations, the high-speed interface 808 is coupled to the memory 804, the display 816 (e.g., through a graphics processor or accelerator), and to the high-speed expansion ports 810, which may accept various expansion cards (not shown). In the implementation, the low-speed interface 812 is coupled to the storage device 806 and the low-speed expansion port 814. The low-speed expansion port 814, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 820, or multiple times in a group of such servers. In addition, it may be implemented in a personal computer such as a laptop computer 822. It may also be implemented as part of a rack server system 824. Alternatively, components from the computing device 800 may be combined with other components in a mobile device (not shown), such as a mobile computing device 850. Each of such devices may contain one or more of the computing device 800 and the mobile computing device 850, and an entire system may be made up of multiple computing devices communicating with each other.

The mobile computing device 850 includes a processor 852, a memory 864, an input/output device such as a display 854, a communication interface 866, and a transceiver 868, among other components. The mobile computing device 850 may also be provided with a storage device, such as a micro-drive or other device, to provide additional storage. Each of the processor 852, the memory 864, the display 854, the communication interface 866, and the transceiver 868, are interconnected using various buses, and several of the components may be mounted on a common motherboard or in other manners as appropriate.

The processor 852 can execute instructions within the mobile computing device 850, including instructions stored in the memory 864. The processor 852 may be implemented as a chipset of chips that include separate and multiple analog and digital processors. The processor 852 may provide, for example, for coordination of the other components of the mobile computing device 850, such as control of user interfaces, applications run by the mobile computing device 850, and wireless communication by the mobile computing device 850.

The processor 852 may communicate with a user through a control interface 858 and a display interface 856 coupled to the display 854. The display 854 may be, for example, a TFT (Thin-Film-Transistor Liquid Crystal Display) display or an OLED (Organic Light Emitting Diode) display, or other appropriate display technology. The display interface 856 may include appropriate circuitry for driving the display 854 to present graphical and other information to a user. The control interface 858 may receive commands from a user and convert them for submission to the processor 852. In addition, an external interface 862 may provide communication with the processor 852, so as to enable near area communication of the mobile computing device 850 with other devices. The external interface 862 may provide, for example, for wired communication in some implementations, or for wireless communication in other implementations, and multiple interfaces may also be used.

The memory 864 stores information within the mobile computing device 850. The memory 864 can be implemented as one or more of a computer-readable medium or media, a volatile memory unit or units, or a non-volatile memory unit or units. An expansion memory 874 may also be provided and connected to the mobile computing device 850 through an expansion interface 872, which may include, for example, a SIMM (Single In Line Memory Module) card interface. The expansion memory 874 may provide extra storage space for the mobile computing device 850, or may also store applications or other information for the mobile computing device 850. Specifically, the expansion memory 874 may include instructions to carry out or supplement the processes described above, and may include secure information also. Thus, for example, the expansion memory 874 may be provide as a security module for the mobile computing device 850, and may be programmed with instructions that permit secure use of the mobile computing device 850. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. That the instructions, when executed by one or more processing devices (for example, processor 852), perform one or more methods, such as those described above. The instructions can also be stored by one or more storage devices, such as one or more computer- or machine-readable mediums (for example, the memory 864, the expansion memory 874, or memory on the processor 852). In some implementations, the instructions can be received in a propagated signal, for example, over the transceiver 868 or the external interface 862.

The mobile computing device 850 may communicate wirelessly through the communication interface 866, which may include digital signal processing circuitry where necessary. The communication interface 866 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 868 using a radio-frequency. In addition, short-range communication may occur, such as using a Bluetooth, WiFi, or other such transceiver (not shown). In addition, a GPS (Global Positioning System) receiver module 870 may provide additional navigation- and location-related wireless data to the mobile computing device 850, which may be used as appropriate by applications running on the mobile computing device 850.

The mobile computing device 850 may also communicate audibly using an audio codec 860, which may receive spoken information from a user and convert it to usable digital information. The audio codec 860 may likewise generate audible sound for a user, such as through a speaker, e.g., in a handset of the mobile computing device 850. Such sound may include sound from voice telephone calls, may include recorded sound (e.g., voice messages, music files, etc.) and may also include sound generated by applications operating on the mobile computing device 850.

The mobile computing device 850 may be implemented in a number of different forms, as shown in the figure. For example, it may be implemented as a cellular telephone 880. It may also be implemented as part of a smart-phone 882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

### Functionalization

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### OTHER EMBODIMENTS AND EQUIVALENTS

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

We claim:

1. A system comprising:
a particle detection apparatus;
a processor; and
a memory storing instructions, wherein the instructions, when executed, cause the processor to:
access data regarding a plurality of events, wherein the plurality of events were detected by the particle detection apparatus;
identify a plurality of groups in the plurality of events, wherein
each of the plurality of groups comprises one or more events, and
a first event of the one or more events comprises at least one measurement selected from the group consisting of: a time duration, a fluorescence intensity, a scatter measurement, a radio frequency signal, a magnetic signal, a neutron scattering measurement, a light scattering measurement, an electron scattering measurement, an audio signal, an acoustic signal, a mechanical signal, an electrical resistance, a thermal property, and a width of fluorescence signal,
wherein the particle detection apparatus collected the measurement; and
identify, based at least in part on the at least one measurement associated with each group of the plurality of groups, a subset of the plurality of groups as a plurality of objects.

2. The system of claim 1, wherein the plurality of objects comprise at least one of a plurality of cells, a plurality of DNA fragments, a plurality of RNA fragments, a plurality of protein aggregates, a plurality of nanostructures, and a plurality of living organisms.

3. The system of claim 1, wherein each object of the plurality of objects is composed at least in part of one or more of a) hydrogel, b) metal, c) glass, and d) plastic.

4. The system of claim 1, wherein the plurality of objects comprise a plurality of encoded objects.

5. The system of claim 4, wherein:
each event of the plurality of events comprises one or more measurements, wherein
the one or more measurements were obtained by the particle detection apparatus, and
a first measurement of the one or more measurements comprises a measurement of a signal encoded to emanate from each object of at least a portion of the plurality of encoded objects; and
identifying a first group of the plurality of groups comprises identifying at least a first region of a particular object and a second region of the particular object, wherein a plurality of signals emanate from two or more spatially separated regions of the particular object.

6. The system of claim 5, wherein:
the plurality of encoded objects comprise a first object type and a second object type; and
identifying the subset of the plurality of groups comprises identifying a second subset of the plurality of groups, wherein at least one region of the encoded objects of the subset of the plurality of groups varies in one or more physical characteristics from a corresponding region of the encoded objects of the second subset of the plurality of groups, wherein
the at least one region varies at a discrete level, allowing the first object type to be reliably distinguished from the second object type based upon the data.

7. The system of claim 5, wherein the signal comprises a light signal.

8. The system of claim 5, wherein the instructions, when executed, cause the processor to quantify a signal associated with a third region of the particular object, wherein the third region is a probe region of the particular object.

9. The system of claim 8, wherein the third region is the first region.

10. The system of claim 5, wherein identifying the first group comprises one or more of:
(a) comparing a time interval between a pair of events of the plurality of events with an expected interval, wherein the expected interval is based at least in part on a combination of (i) a flow velocity setting of the particle detection apparatus at time of detection, and (ii) a physical distance between a pair of event sources on the particular object;
(b) comparing the duration of a first event of the plurality of events with an expected duration, wherein the expected duration is based at least in part on a combination of i) the velocity setting of the particle detection apparatus at time of detection, and (ii) a physical dimension of a first event source on the particular object;
(c) comparing fluorescence intensities of a sequence of two events of the plurality of events with an expected sequence of fluorescence intensities, wherein the expected sequence of fluorescence intensities is based at least in part on optical characteristics of the particular object; and
(d) comparing scattering intensities of a sequence of two events of the plurality of events with an expected sequence of scattering intensities, wherein the expected sequence of scattering intensities is based at least in part on optical characteristics of the particular object.

11. The system of claim 1, wherein:
a first measurement of the at least one measurement comprises a measurement of a signal encoded to emanate from each object of at least a portion of the plurality of objects; and
identifying a first group of the plurality of groups comprises identifying at least a first region of a particular object and a second region of the particular object, wherein a plurality of signals emanate from two or more spatially separated regions of the particular object.

12. The system of claim 11, wherein:
the plurality of objects comprise a first object type and a second object type; and
identifying the subset of the plurality of groups comprises identifying a second subset of the plurality of groups, wherein at least one region of the objects of the subset of the plurality of groups varies in one or more physical characteristics from a corresponding region of the objects of the second subset of the plurality of groups, wherein
the at least one region varies at a discrete level, allowing the first object type to be reliably distinguished from the second object type based upon the data.

13. The system of claim 12, wherein two or more predetermined sets of levels are combined into codes for encoding the plurality of objects, thereby allowing a plurality of different sets of objects to be identified.

14. The system of claim 1, wherein the plurality of objects comprise a plurality of carriers brought in contact with a sample comprising an analyte prior to detection by the particle detection apparatus.

15. The system of claim 14, wherein the analyte comprises a protein or a nucleic acid.

16. The system of claim 14, wherein one or more of the measurements associated with each object of the plurality of objects are indicative of a concentration of the analyte within the sample.

17. The system of claim 16, wherein:
identifying the subset of the plurality of groups as the plurality of objects comprises identifying the plurality of objects as being a type of object sensitive to the analyte; and wherein
the instructions, when executed, cause the processor to determine the concentration of the analyte, wherein the concentration of the analyte is determined by statistical analysis of the one or more measurements associated with each object of the plurality of objects.

18. The system of claim 17, wherein:
two or more different carriers are brought in contact with the sample simultaneously; and
determining the concentration of the analyte comprises determining respective concentrations of two or more analytes.

19. The system of claim 17, wherein the statistical analysis includes the calculation of one or more of the following: mean, median, standard deviation and confidence intervals.

20. The system of claim 17, wherein the instructions, when executed, cause the processor to, prior to determining the concentration of the analyte:
identify one or more outlier measurements of the one or more measurements associated with the plurality of objects; and
remove the one or more outlier measurements from a set of measurements provided for statistical analysis.

21. The system of claim 20, wherein identifying the one or more outlier measurements comprises:
ordering all measurements; and
selecting a lower percentile and upper percentile.

22. The system of claim 1, wherein the instructions, when executed, cause the processor to:
determine, for each object of the plurality of objects, based in part upon respective one or more quantities associated with the respective object, information regarding a history of the respective object.

23. The system of claim 22, wherein the history of the respective object is determined at least in part by a physical, chemical or biological assay.

24. The system of claim 1, wherein the particle detection apparatus comprises at least one of a flow cytometer, a particle counter, a Coulter counter, a microarray scanner, and a plate imager.

25. A method comprising:
accessing data regarding a plurality of events, wherein the plurality of events were detected by a particle detection apparatus;
identifying, by a processor of a computing device, a plurality of groups in the plurality of events, wherein
each of the plurality of groups comprises two or more events, and
each event of the plurality of events comprises one or more of a time stamp, a time duration, a fluorescence intensity, a scatter measurement, a radio frequency signal, a magnetic signal, a neutron scattering measurement, a light scattering measurement, an electron scattering measurement, an audio signal, an acoustic signal, a mechanical signal, an electrical resistance, a thermal property, and a width of fluorescence signal; and
identifying, by the processor, a subset of the plurality of groups as a plurality of objects detected by the particle detection apparatus, wherein
each object of the plurality of objects is identified based at least in part upon one or more quantities, wherein each quantity of the one or more quantities is identified by or derived from the respective two or more events.

26. The method of claim 25, wherein identifying a first object of the plurality of objects comprises:
(a) defining a fit-function F of a plurality of measurements, wherein
the plurality of measurements are obtained from the plurality of events, and
the fit-function F is configured to evaluate the correspondence of each event of the plurality of events with known physical characteristics of the particular object;
(b) selecting, from the plurality of events, a subset of the plurality of events which optimizes the fit-function F, wherein the subset of the plurality of events is selected as a particular group of events most likely to originate from a same physical object of the plurality of objects; and
(c) assigning a score to a fit identified by the fit-function F, wherein the score is configured to assess a probability of error in selecting the correct subset of the plurality of events.

27.-35. (canceled)

36. The method of claim 26, wherein identifying the plurality of groups comprises:
(a) out of a first Nᵣ+g consecutive unassigned events of the plurality of events, starting with a consecutive event following a last unassigned event of the plurality of events, selecting all combinations of Nᵣ events, wherein a gap count g indicates a number of allowed gaps, wherein the gap count g is configured to range from zero to any positive integer;
(b) calculating, for each selected combination of Nᵣ events, the fit function F with respective candidate regions of respective candidate combinations of events assigned to events in order of increasing time to identify respective forward direction fits;
(c) calculating, for each selected combination of Nᵣ events, the fit function F with respective candidate regions of respective candidate combinations of events assigned to events in order of decreasing time to identify respective reverse direction fits;
(d) identifying, from the forward direction fits and the reverse direction fits, i) a lowest fit combination of the selected combination of Nᵣ events and ii) a respective direction of the lowest fit combination, wherein the events of the lowest fit combination are assigned to respective regions of the object according to the direction of the lowest fit combination; and
(e) repeating steps (a) through (d) until a remaining number of events after the last assigned event is less than Nᵣ.

37. The method of claim 25, comprising identifying an orientation of each object of the plurality of objects.

38. The method of claim 25, wherein the particle detection apparatus comprises standard flow cytometry instrumentation.

39. The method of claim 25, wherein the data comprises a file in the standard flow cytometry format (FCS).

40. A non-transitory computer-readable medium having instructions stored thereon, wherein the instructions, when executed by a processor, cause the processor to:
access data regarding a plurality of events, wherein the plurality of events were detected by a particle detection apparatus;
identify a plurality of groups in the plurality of events, wherein
each of the plurality of groups comprises one or more events, and
a first event of the one or more events comprises at least one measurement selected from the group consisting of: a time duration, a fluorescence intensity, a scatter measurement, a radio frequency signal, a magnetic signal, a neutron scattering measurement, a light scattering measurement, an electron scattering measurement, an audio signal, an acoustic signal, a mechanical signal, an electrical resistance, a thermal property, and a width of fluorescence signal, wherein the particle detection apparatus collected the measurement; and
identify, based at least in part on the at least one measurement associated with each group of the plurality of groups, a subset of the plurality of groups as a plurality of objects.
